(19) United States
(12) Patent Application Publication
(10) Pub. No.: US 2012/0004222 A1
(43) Pub. Date: Jan. 5, 2012

(54) CB2 RECEPTOR LIGANDS FOR THE TREATMENT OF PAIN

(76) Inventors: Zhicai Wu, Montvale, NJ (US); Ahren I. Green, Penllyn, PA (US); Jonh C. Hartnett, Philadelphia, PA (US)

(21) Appl. No.: 12/672,752

(22) PCT Filed: Aug. 18, 2008
(86) PCT No.: PCT/US08/09842

§ 371 Date: Feb. 9, 2010

Related U.S. Application Data
(60) Provisional application No. 60/965,561, filed on Aug. 21, 2007.

(51) Int. Cl. A61K 31/5377; A61K 31/4545; A61P 19/02; A61P 19/10; A61P 9/10; C07D 471/04; A61P 29/00

(52) U.S. Cl. 514/233.2; 544/127; 546/121; 514/300

(57) ABSTRACT

The present invention relates to compounds represented by Formula (I) and Formula (II):

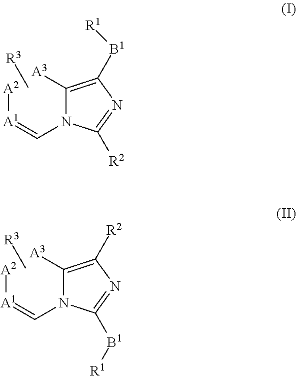

or pharmaceutically acceptable salts thereof. The present invention also provides pharmaceutical compositions comprising the instant compounds. This invention further provides methods to treat and prevent pain, respiratory and non-respiratory diseases.

### FIELD OF THE INVENTION

This invention relates to compounds useful as cannabinoid receptor modulators. More particularly, this invention relates to compounds that are CB2 modulators. Even more particularly, this invention relates to compounds that are selective CB2 agonists. Selective CB2 agonists are expected to be devoid of psychotropic effects present in non-selective cannabinoids attributed to interaction with the CB1 receptor. The compounds of the invention are useful in the treatment of pain in a range of pain states, such as inflammatory and neuropathic pain.

### BACKGROUND OF THE INVENTION

Cannabinoids are psychoactive natural products present in Cannabis sativa L. and have been used as therapeutic agents for thousands of years. They have been shown to have myriad effects in humans, notably in the central nervous system and the cardiovascular system. The therapeutic utility of Cannabis is significantly limited due to adverse central effects. The effects of cannabinoids have been shown to occur through their action on two G-protein coupled receptors. A first receptor, CB1, is primarily a centrally-expressed receptor with more limited expression in a variety of peripheral sites, and is believed to be primarily responsible for the central effects of cannabinoids. A second receptor, CB2, is preferentially expressed in the periphery, primarily in cells of the immune system, although it has been identified in central locations to a lesser extent. CB2, expressed in immune cells such as T-cells, B-cells, macrophages and mast cells, has been shown to have a specific role in mediating immune and inflammatory responses. Given the role of the CB2 receptor in immunomodulation, it is an attractive target for chronic inflammatory pain. CB2 modulators also may have a role in the treatment of osteoporosis, atherosclerosis, immune disorders, arthritis and other pathological conditions, as discussed infra.

The effects of cannabinoids are due to interaction with specific high affinity receptors, coupled to G-proteins, present at the central level (Devane et al., Molecular Pharmacology (1988), 34, 605-613) and the peripheral level (Nye et al., J. Pharmacol. and Exp. Ther. (1985), 234, 784-791; Kaminski et al., Molecular Pharmacol. (1992), 42, 736-742; Munro et al., Nature (1993), 365, 61-65).

The central effects of cannabinoids relate to a first type of cannabinoid receptor (CB1) which is present mainly in the brain, but also in the periphery. Munro et al. [Nature, (1993) 365, 61-65] have cloned a second type of cannabinoid receptor, CB2, which is present in the periphery and more particularly on cells of immune origin. The presence of CB2 cannabinoid receptors on lymphoid cells may explain the immunomodulation mentioned above exerted by agonists for cannabinoid receptors.

The psychotropic effects of cannabinoids as well as their influence on immune function have been described. [Hollister, L. E., J. Psychoact. Drugs, 24 (1992), 159-164]. Most of the in vitro studies have shown immunosuppressant effects for cannabinoids: the inhibition of the proliferative responses in T-lymphocytes and B-lymphocytes induced by mitogens [Luo, Y. D. et al., Int. J. Immunopharmacol., (1992) 14, 49-56, Schwartz, H. et al., J. Neuroimmunol., (1994) 55, 107-115], the inhibition of the activity of cytotoxic T-cells [Klein et al., J. Toxicol. Environ. Health, (1991) 32, 465-477], the inhibition of the microbiocidal activity of macrophages and of the synthesis of TNF-α. [Arata, S. et al., Life Sci., (1991) 49, 473-479; Fisher-Stenger et al., J. Pharm. Exp. Ther., (1993) 267, 1558-1565], the inhibition of the cytolytic activity and of the production of TNF-α. of large granular lymphocytes [Kusher et al., Cell. Immun., (1994) 154, 99-108]. In some studies, amplification effects were observed: increase in the bioactivity of interleukin-1 by mice resident macrophages or differentiated macrophage cell lines, due to increased levels of TNF-α. [Zhu et al., J. Pharm. Exp. Ther., (1994) 270, 1334-1339; Shivers, S. C. et al., Life Sci., (1994) 54, 1281-1289].

Certain Imadazo[1,5-a]pyridine analogs have been disclosed as useful for the inhibition of fibroblast growth factor. See WO2006097625, published Sep. 21, 2006.

### SUMMARY OF THE INVENTION

The present invention relates to compounds represented by Formula (I) and Formula (II):

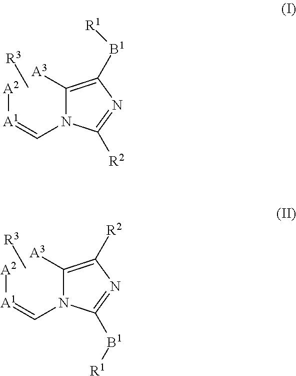

or pharmaceutically acceptable salts thereof. The present invention also provides pharmaceutical compositions comprising the instant compounds. This invention further provides methods to treat and prevent pain, osteoarthritis, atherosclerosis, Multiple Sclerosis, Alzheimer's, and respiratory and non-respiratory diseases.

### DETAILED DESCRIPTION OF THE INVENTION

In one embodiment the present invention relates to compounds represented by Formula (I) and Formula (II):

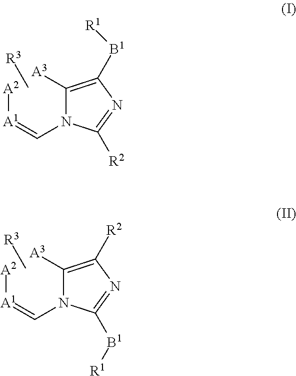

and pharmaceutically acceptable salts thereof, wherein
A¹, A², and A³ are selected from the group consisting of:

### (1) CH and

### (2) N;

B¹ is aryl or heteroaryl;
R¹ and R² are independently selected from the group consisting of:
   (1) H,
   (2) halo,
   (3) —CN,
   (4) —CF₃,
   (5) —C₁₋₆alkyl,
   (6) —C(O)—NH—C₁₋₃alkyl-CF₃,
   (7) —C(O)—NH—C₁₋₃allyl-heteroaryl, wherein the heteroaryl is optionally mono, di or tri-substituted with substituents independently selected from R⁶,
   (8) —C(O)-heteroaryl, wherein the heteroaryl is optionally mono, di or tri-substituted with substituents independently selected from R⁶,
   (9) —C(O)-heterocycle, wherein the heterocycle is optionally mono, di- or tri-substituted with substituents independently selected from R⁶,
   (10) —NR⁴R⁵,
   (11) —C₁₋₄allyl-NR⁴R⁵,
   (12) —C₁₋₄allyl-heterocycle, wherein the alkyl is optionally substituted with hydroxyl and wherein the heterocycle is optionally mono, di- or tri-substituted with substituents independently selected from R⁶,
   (13) —C₁₋₄alkyl-heteroaryl, optionally mono, di- or tri-substituted with substituents independently selected from R⁶,
   (14) heterocycle, optionally mono, di- or tri-substituted with substituents independently selected from R⁶,
   (15) heteroaryl, optionally mono, di- or tri-substituted with substituents independently selected from R⁶,
   (16) aryl, optionally mono, di- or tri-substituted with substituents independently selected from R⁷,
   (17) O-aryl, optionally mono, di- or tri-substituted with substituents independently selected from R⁷,
   (18) —O-heteroaryl, optionally mono, di- or tri-substituted with substituents independently selected from R⁶,
   (19) —NH-heteroaryl, wherein the heteroaryl is optionally mono, di- or tri-substituted with substituents independently selected R⁶, and
   (20) —C₃₋₆cycloalkyl, optionally mono, di- or tri-substituted with substituents selected —CH₃, —O—C₁₋₆alkyl, hydroxy, —CH₂—OH, halo, —S(O)₂—CH₃, —C(O)—O—C₁₋₆alkyl, —C(O)—NHC₁₋₆allyl, —C(O)—N(C₁₋₆alkyl)₂, oxo, C(O)—O—C(CH₃)₃, —C₃₋₆cycloallyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN;
R³ is selected from the group consisting of:
   (1) H,
   (2) halo,
   (3) —C₁₋₄alkyl, optionally substituted with hydroxyl,
   (4) —CF₃, and
   (5) —OC₁₋₆alkyl;
   (6) —CN,
   (7) —CHF₂,
   (8) —O—CF₃,
   (9) hydroxy,
   (10) —S(O)₂—CH₃,
   (11) —C(O)—O—C₁₋₆allyl,
   (12) —C(O)—NHC₁₋₆allyl,
   (13) —C(O)—N(C₁₋₆allyl)₂,
   (14) —C(O)—O—C(CH₃)₃,
   (15) —C(O)-heteroaryl,
   (16) —C₃₋₆cycloalkyl,
   (17) —NH₂,
   (18) —NH₂—C(O)—CF₃,
   (19) —NH₂—C(O)—N(CH₃)₂,
   (20) —NC(O)—NH₂,
   (21) —NH—S(O)₂—CH₃,
   (22) heteroaryl, optionally mono, di- or tri-substituted with substituents independently selected from R⁶, and
   (23) aryl, optionally mono, di- or tri-substituted with substituents independently selected from R⁷;
R⁴ is selected from the group consisting of hydrogen and methyl;
R⁵ is selected from the group consisting of:
   (1) C₁₋₄alkyl, optionally mono or di-substituted, with substituents independently selected from the group consisting of C₃₋₆cycloalkyl, —CF₃, heteroaryl, —C₁₋₃alkyl-CF₃, CH₃, hydroxy, tetrahydrofuran, and
   (2) —C₁₋₃alkyl-C₃₋₆cycloalkyl, wherein the cycloalkyl is optionally mono or di-substituted with substituents independently selected from the group consisting of halo, CF₃, CH₃, C₁₋₃alkyl, —OC₁₋₆alkyl, or
R⁴ and R⁵ are joined together so that along with the nitrogen to which they are attached, there is formed a heterocycle, wherein said heterocycle is optionally mono, di or tri-substituted with substituents independently selected from the group consisting of —OC₁₋₆alkyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)₂, oxo, —C(O)—O—C(CH₃)₃, —C(O)-heteroaryl, —C₃₋₆cycloallyl, —NH₂, —NH—C(O)—CF₃, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆alkyl;
R⁶ is selected from the group consisting of —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆allyl;
R⁷ is selected from the group consisting of —C₁₋₁₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆allyl)₂, —(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl; provided that when the compound is of Formula (II), and B¹ is optionally substituted aryl, then R² is other than hydrogen, halo, cyano, optionally substituted aryl, optionally substituted heteroaryl or NR⁴R⁵ wherein both of R⁴ and R⁵ are hydrogen, or unsubstituted alkyl.
Within this embodiment there is a genus wherein
R¹ is selected from the group consisting of:
   (1) H,
   (2) halo,
   (3) —CN,
   (4) —CF₃,
   (5) —C₁₋₆alkyl,
   (6) —C(O)—NH—C₁₋₃alkyl-CF₃,
   (7) —C(O)—NH—C₁₋₃alkyl-heteroaryl, wherein the heteroaryl is optionally mono, di or tri-substituted with substituents independently selected from R⁶,
   (8) heterocycle, optionally mono, di- or tri-substituted with substituents independently selected from R⁶,
   (9) heteroaryl, optionally mono, di- or tri-substituted with substituents independently selected from R⁶,
   (10) aryl, optionally mono, di- or tri-substituted with substituents independently selected from R⁷,
   (11) O-aryl, optionally mono, di- or tri-substituted with substituents independently selected from R⁷,
   (12) —O-heteroaryl, optionally mono, di- or tri-substituted with substituents independently selected from R⁶, and
   (13) —NH-heteroaryl, wherein the heteroaryl is optionally mono, di- or tri-substituted with substituents independently selected R⁶.
Within this embodiment there is a genus wherein
R² is selected from a group consisting of:
   (1) halo,
   (2) —CF₃,
   (3) —C(O)—NH—C₁₋₃alkyl-CF₃,
   (4) —C(O)—NH—C₁₋₃alkyl-heteroaryl, wherein the heteroaryl is optionally mono, di or tri-substituted with substituents independently selected from R⁶,
   (5) —C(O)-heteroaryl, wherein the heteroaryl is optionally mono, di or tri-substituted with substituents independently selected from R⁶,
   (6) —C(O)-heterocycle, wherein the heterocycle is optionally mono, di- or tri-substituted with substituents independently selected from R⁶,
   (7) —NR⁴R⁵,
   (8) —C₁₋₄alkyl-NR⁴R⁵,
   (9) —C₁₋₄alkyl-heterocycle, wherein the alkyl is optionally substituted with hydroxyl and wherein the heterocycle is optionally mono, di- or tri-substituted with substituents independently selected from R⁶,
   (10) —C₁₋₄alkyl-heteroaryl, optionally mono, di- or tri-substituted with substituents independently selected from R⁶,
   (11) heterocycle, optionally mono, di- or tri-substituted with substituents independently selected from R⁶,
   (12) heteroaryl, optionally mono, di- or tri-substituted with substituents independently selected from R⁶,
   (13) aryl, optionally mono, di- or tri-substituted with substituents independently selected from R⁷,
   (14) O-aryl, optionally mono, di- or tri-substituted with substituents independently selected from R⁷,
   (15) —O-heteroaryl, optionally mono, di- or tri-substituted with substituents independently selected from R⁶,
   (16) —NH-heteroaryl, wherein the heteroaryl is optionally mono, di- or tri-substituted with substituents independently selected R⁶, and
   (17) —C₃₋₆cycloalkyl, optionally mono, di- or tri-substituted with substituents selected —CH₃, —O—C₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, oxo, C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN.
Within this genus there is a sub-genus wherein
R² is selected from the group consisting of:
   (1) —CF₃,
   (2) —NR⁴R⁵,
   (3) —C₁₋₄alkyl-NR⁴R⁵,
   (4) —C₁₋₄alkyl-heterocycle, wherein the alkyl is optionally substituted with hydroxyl and wherein the heterocycle is optionally mono, di- or tri-substituted with substituents independently selected from R⁶, and
   (5) heterocycle, optionally mono, di- or tri-substituted with substituents independently selected from R⁶.
Within this embodiment there is a genus wherein
R³ is selected from the group consisting of:
   (1) H,
   (2) halo,
   (3) —C₁₋₄alkyl, optionally substituted with hydroxyl,
   (4) —CF₃, and
   (5) —OC₁₋₆alkyl;
   (6) —CN,
   (7) —CHF₂,
   (8) —O—CF₃,
   (9) —S(O)₂—CH₃,
   (10) —C(O)—O—C₁₋₆alkyl,
   (11) —C(O)—NHC₁₋₆alkyl,
   (12) —C(O)—N(C₁₋₆alkyl)₂,
   (13) —C(O)—O—C(CH₃)₃,
   (14) —C(O)-heteroaryl,
   (15) —C₃₋₆cycloalkyl,
   (16) —NH₂—C(O)—CF₃,
   (17) —NH₂—C(O)—N(CH₃)₂,
   (18) —NC(O)—NH₂, and
   (19) —NH—S(O)₂—CH₃.
Within this genus there is a sub-genus wherein
R³ is sleeted from a group consisting of:
   (1) H,
   (2) halo,
   (3) —C₁₋₄alkyl, optionally substituted with hydroxyl,
   (4) —CF₃, and
   (5) —CN,
   (6) —S(O)₂—CH₃,
   (7) —C(O)—NHC₁₋₆alkyl,
   (8) —C(O)—N(C₁₋₆alkyl)₂,
   (9) —NH₂—C(O)—CF₃,
   (10) —NH₂—C(O)—N(CH₃)₂,
   (11) —NC(O)—NH₂, and
   (12) —NH—S(O)₂—CH₃.
Within this embodiment there is a genus wherein
R⁴ and R⁵ are joined together so that along with the nitrogen to which they are attached, there is formed a heterocycle, wherein said heterocycle is optionally mono, di or tri-substituted with substituents independently selected from the group consisting of —OC₁₋₆alkyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)₂, oxo, —C(O)—O—C(CH₃)₃, —C(O)-heteroaryl, —C₃₋₆cycloalkyl, —NH₂, —NH—C(O)—CF₃, —C(O)—NHC₁₋₆allyl, —C(O)—N(C₁₋₆alkyl)₂, —NC(O)—NH₂, —NH—S(O)₂—CH₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆alkyl.
Within this embodiment there is a genus wherein
A¹, A², and A³ are selected from the group consisting of:

### (1) CH and

### (2) N;

B¹ is aryl or heteroaryl;
R¹ is selected from the group consisting of:
   (1) H,
   (2) halo,
   (3) —CN,
   (4) —CF₃,
   (5) —C₁₋₆allyl,
   (6) —C(O)—NH—C₁₋₃alkyl-CF₃,
   (7) —C(O)—NH—C₁₋₃allyl-heteroaryl, wherein the heteroaryl is optionally mono, di or tri-substituted with substituents independently selected from R⁶,
   (8) heterocycle, optionally mono, di- or tri-substituted with substituents independently selected from R⁶,
   (9) heteroaryl, optionally mono, di- or tri-substituted with substituents independently selected from R⁶,
   (10) aryl, optionally mono, di- or tri-substituted with substituents independently selected from R⁷,
   (11) O-aryl, optionally mono, di- or tri-substituted with substituents independently selected from R⁷,
   (12) —O-heteroaryl, optionally mono, di- or tri-substituted with substituents independently selected from R⁶, and
   (13) —NH-heteroaryl, wherein the heteroaryl is optionally mono, di- or tri-substituted with substituents independently selected R⁶_{;}
R² is selected from a group consisting of:
   (1) halo,
   (2) —CF₃,
   (3) —C(O)—NH—C₁₋₃alkyl-CF₃,
   (4) —C(O)—NH—C₁₋₃alkyl-heteroaryl, wherein the heteroaryl is optionally mono, di or tri-substituted with substituents independently selected from R⁶,
   (5) —C(O)-heteroaryl, wherein the heteroaryl is optionally mono, di or tri-substituted with substituents independently selected from R⁶,
   (6) —C(O)-heterocycle, wherein the heterocycle is optionally mono, di- or tri-substituted with substituents independently selected from R⁶,
   (7) —NR⁴R⁵,
   (8) —C₁₋₄allyl-NR⁴R⁵,
   (9) —C₁₋₄alkyl-heterocycle, wherein the alkyl is optionally substituted with hydroxyl and wherein the heterocycle is optionally mono, di- or tri-substituted with substituents independently selected from R⁶,
   (10) —C₁₋₄alkyl-heteroaryl, optionally mono, di- or tri-substituted with substituents independently selected from R⁶,
   (11) heterocycle, optionally mono, di- or tri-substituted with substituents independently selected from R⁶,
   (12) heteroaryl, optionally mono, di- or tri-substituted with substituents independently selected from R⁶,
   (13) aryl, optionally mono, di- or tri-substituted with substituents independently selected from R⁷,
   (14) O-aryl, optionally mono, di- or tri-substituted with substituents independently selected from R⁷,
   (15) —O-heteroaryl, optionally mono, di- or tri-substituted with substituents independently selected from R⁶,
   (16) —NH-heteroaryl, wherein the heteroaryl is optionally mono, di- or tri-substituted with substituents independently selected R⁶, and
   (17) —C₃₋₆cycloalkyl, optionally mono, di- or tri-substituted with substituents selected —CH₃, —O—C₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, oxo, C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN;
R³ is selected from the group consisting of:
   (1) H,
   (2) halo,
   (3) —C₁₋₄allyl, optionally substituted with hydroxyl,
   (4) —CF₃, and
   (5) —OC₁₋₆allyl;
   (6) —CN,
   (7) —CHF₂,
   (8) —O—CF₃,
   (9) —S(O)₂—CH₃,
   (10) —C(O)—O—C₁₋₆alkyl,
   (11) —C(O)—NHC₁₋₆allyl,
   (12) —C(O)—N(C₁₋₆alkyl)₂,
   (13) —C(O)—O—C(CH₃)₃,
   (14) —C(O)-heteroaryl,
   (15) —C₃₋₆cycloalkyl,
   (16) —NH₂—C(O)—CF₃,
   (17) —NH₂—C(O)—N(CH₃)₂,
   (18) —NC(O)—NH₂, and
   (19) —NH—S(O)₂—CH₃;
R⁴ is selected from the group consisting of hydrogen and methyl;
R⁵ is selected from the group consisting of:
   (1) C₁₋₄alkyl, optionally mono or di-substituted, with substituents independently selected from the group consisting of C₃₋₆cycloalkyl, —CF₃, heteroaryl, —C_{1 —} 3alkyl-CF₃, CH₃, hydroxy, tetrahydrofuran, and
   (2) —C₁₋₃alkyl-C₃₋₆cycloalkyl, wherein the cycloalkyl is optionally mono or di-substituted with substituents independently selected from the group consisting of halo, CF₃, CH₃, C₁₋₃alkyl, —OC₁₋₆alkyl, or
R⁴ and R⁵ are joined together so that along with the nitrogen to which they are attached, there is formed a heterocycle, wherein said heterocycle is optionally mono, di or tri-substituted with substituents independently selected from the group consisting of —OC₁₋₆allyl, —NH—C(O)—O—C(CH₃)₃, hydroxy, —CH₃, —CF₃, —CH₂—OH, halo, —S(O)₂—C₁₋₁₃, —C(O)—N(CH₃)₂, oxo, —C(O)—O—C(CH₃)₃, —C(O)-heteroaryl, —C₃₋₆cycloalkyl, —NH₂, —NH—C(O)—CF₃, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆alkyl;
R⁶ is selected from the group consisting of —CN, —CH₃, —CF₃, —CHF₂, —OC₁₋₆alkyl, —O—CF₃, hydroxy, —CH₂—OH, halo, —S(O)₂—CH₃, —C(O)—NHC₁₋₆alkyl, —C(O)—N(C₁₋₆alkyl)₂, —C(O)—O—C(CH₃)₃, —C(O)-heteroaryl, —C₃₋₆cycloallyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl;
R⁷ is selected from the group consisting of —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl;
   provided that when the compound is of Formula (II), and B¹ is optionally substituted aryl, then R² is other than hydrogen, halo, cyano, optionally substituted aryl, optionally substituted heteroaryl or NR⁴R⁵ wherein both of R⁴ and R⁵ are hydrogen, or unsubstituted alkyl.
Within this embodiment there is a genus wherein
A¹, A², and A³ are selected from the group consisting of:

### (1) CH and

### (2) N;

B¹ is aryl or heteroaryl;
R¹ is selected from the group consisting of:
   (1) H,
   (2) halo,
   (3) —CN,
   (4) —CF₃,
   (5) —C₁₋₆alkyl,
   (6) —C(O)—NH—C₁₋₃alkyl-CF₃,
   (7) —C(O)—NH—C₁₋₃allyl-heteroaryl, wherein the heteroaryl is optionally mono, di or tri-substituted with substituents independently selected from R⁶,
   (8) heterocycle, optionally mono, di- or tri-substituted with substituents independently selected from R⁶,
   (9) heteroaryl, optionally mono, di- or tri-substituted with substituents independently selected from R⁶,
   (10) aryl, optionally mono, di- or tri-substituted with substituents independently selected from R⁷,
   (11) O-aryl, optionally mono, di- or tri-substituted with substituents independently selected from R⁷,
   (12) —O-heteroaryl, optionally mono, di- or tri-substituted with substituents independently selected from R⁶, and
   (13) —NH-heteroaryl, wherein the heteroaryl is optionally mono, di- or tri-substituted with substituents independently selected R⁶_{;}
R² is selected from the group consisting of:
   (1) —CF₃,
   (2) —NR⁴R⁵,
   (3) —C₁₋₄alkyl-NR⁴R⁵,
   (4) —C₁₋₄alkyl-heterocycle, wherein the alkyl is optionally substituted with hydroxyl and wherein the heterocycle is optionally mono, di- or tri-substituted with substituents independently selected from R⁶, and
   (5) heterocycle, optionally mono, di- or tri-substituted with substituents independently selected from R⁶_{;}
R³ is sleeted from a group consisting of:
   (1) H,
   (2) halo,
   (3) —C₁₋₄alkyl, optionally substituted with hydroxyl,
   (4) —CF₃, and
   (5) —CN,
   (6) —S(O)₂—CH₃,
   (7) —C(O)—NHC₁₋₆alkyl,
   (8) —C(O)—N(C₁₋₆alkyl)₂,
   (9) —NH₂—C(O)—CF₃,
   (10) —NH₂—C(O)—N(CH₃)₂,
   (11) —NC(O)—NH₂, and
   (12) —NH—S(O)₂—CH₃;
R⁴ and R⁵ are joined together so that along with the nitrogen to which they are attached, there is formed a heterocycle, wherein said heterocycle is optionally mono, di or tri-substituted with substituents independently selected from the group consisting of —OC₁₋₆alkyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)₂, oxo, —C(O)—O—C(CH₃)₃, —C(O)-heteroaryl, —C₃₋₆cycloalkyl, —NH₂, —NH—C(O)—CF₃, —C(O)—NHC₁₋₆allyl, —C(O)—N(C₁₋₆allyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆alkyl;
R⁶ is selected from the group consisting of —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloallyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆allyl;
R⁷ is selected from the group consisting of —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl.

One embodiment of the present invention is a compound of Formula (Ia) and Formula (IIa), wherein:

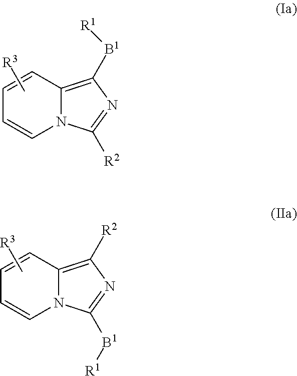

B¹ is aryl or heteroaryl;
R¹ and R² are independently selected from the group consisting of:
   (1) H,
   (2) halo,
   (3) —CN,
   (4) —CF₃,
   (5) —C₁₋₆alkyl,
   (6) —C(O)—NH—C₁₋₃ alkyl-CF₃,
   (7) —C(O)—NH—C₁₋₃alkyl-heteroaryl, wherein the heteroaryl is optionally mono, di or tri-substituted with substituents independently selected from R⁶,
   (8) —C(O)-heteroaryl, wherein the heteroaryl is optionally mono, di or tri-substituted with substituents independently selected from R⁶,
   (9) —C(O)-heterocycle, wherein the heterocycle is optionally mono, di- or tri-substituted with substituents independently selected from R⁶,
   (10) —NR⁴R⁵,
   (11) —C₁₋₄alkyl-NR⁴R⁵,
   (12) —C₁₋₄alkyl-heterocycle, wherein the alkyl is optionally substituted with hydroxyl and wherein the heterocycle is optionally mono, di- or tri-substituted with substituents independently selected from R⁶,
   (13) —C₁₋₄alkyl-heteroaryl, optionally mono, di- or tri-substituted with substituents independently selected from R⁶,
   (14) heterocycle, optionally mono, di- or tri-substituted with substituents independently selected from R⁶,
   (15) heteroaryl, optionally mono, di- or tri-substituted with substituents independently selected from R⁶,
   (16) aryl, optionally mono, di- or tri-substituted with substituents independently selected from R⁷,
   (17) O-aryl, optionally mono, di- or tri-substituted with substituents independently selected from R⁷,
   (18) —O-heteroaryl, optionally mono, di- or tri-substituted with substituents independently selected from R⁶,
   (19) —NH-heteroaryl, wherein the heteroaryl is optionally mono, di- or tri-substituted with substituents independently selected R⁶, and
   (20) —C₃₋₆cycloallyl, optionally mono, di- or tri-substituted with substituents selected —CH₃, —O—C₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, oxo, C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN;
R³ is selected from the group consisting of:
   (1) H,
   (2) halo,
   (3) —C₁₋₄allyl, optionally substituted with hydroxyl,
   (4) —CF₃, and
   (5) —OC₁₋₆allyl;
   (6) —CN,
   (7) —CHF₂,
   (8) —O—CF₃,
   (9) hydroxy,
   (10) —S(O)₂—CH₃,
   (11) —C(O)—O—C₁₋₆alkyl,
   (12) —C(O)—NHC₁₋₆allyl,
   (13) —C(O)—N(C₁₋₆alkyl)₂;
   (14) —C(O)—O—C(CH₃)₃,
   (15) —C(O)-heteroaryl,
   (16) —C₃₋₆cycloalkyl,
   (17) —NH₂,
   (18) —NH₂—C(O)—CF₃,
   (19) —NH₂—C(O)—N(CH₃)₂,
   (20) —NC(O)—NH₂,
   (21) —NH—S(O)₂—CH₃,
   (22) heteroaryl, optionally mono, di- or tri-substituted with substituents independently selected from R⁶, and
   (23) aryl, optionally mono, di- or tri-substituted with substituents independently selected from R⁷;
R⁴ is selected from the group consisting of hydrogen and methyl;
R⁵ is selected from the group consisting of:
   (1) C₁₋₄alkyl, optionally mono or di-substituted, with substituents independently selected from the group consisting of C₃₋₆cycloalkyl, —CF₃, heteroaryl, —C₁₋₃alkyl-CF₃, CH₃, hydroxy, tetrahydrofuran, and
   (2) —C₁₋₃alkyl-C₃₋₆cycloalkyl, wherein the cycloalkyl is optionally mono or di-substituted with substituents independently selected from the group consisting of halo, CF₃, CH₃, C₁₋₃alkyl, —OC₁₋₆alkyl, or
R⁴ and R⁵ are joined together so that along with the nitrogen to which they are attached, there is formed a heterocycle, wherein said heterocycle is optionally mono, di or tri-substituted with substituents independently selected from the group consisting of —OC₁₋₆alkyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)₂, oxo, —C(O)—O—C(CH₃)₃, —C(O)-heteroaryl, —C₃₋₆cycloalkyl, —NH₂, —NH—C(O)—CF₃, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆allyl;
R⁶ is selected from the group consisting of —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloallyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl;
R⁷ is selected from the group consisting of —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl;
   provided that when the compound is of Formula (II), and B¹ is optionally substituted aryl, then R² is other than hydrogen, halo, cyano, optionally substituted aryl, optionally substituted heteroaryl or NR⁴R⁵ wherein both of R⁴ and R⁵ are hydrogen, or unsubstituted alkyl.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

As used here a “cycloalkyl”, is a saturated monocyclic hydrocarbon ring.

As used here a “carbocycle”, is a mono cyclic or bi-cyclic carbocyclic non-aromatic ring having at least one double bond.

The term “aryl”, unless specifically stated otherwise, refers to single and multi-cyclic aromatic ring systems in which the ring members are all carbon, for example, phenyl or naphthyl.

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Lines drawn into the ring systems from substituents indicate that the indicated bond can be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups can be on the same carbon or on different carbons, so long as a stable structure results. The phrase “optionally substituted with one or more substituents” should be taken to be equivalent to the phrase “optionally substituted with at least one substituent” and in such cases one embodiment will have from zero to three substituents.

The term “heteroaryl”, unless specifically stated otherwise, refers to single and multi-cyclic aromatic ring systems in which at least one of the ring members is other than carbon. Heteroaryl includes, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridine, pyridazine, pyrimidine, pyrazine, triazine, including 1,2,4-triazine and 1,3,5-triazine, tetrazine, including 1,2,4,5-tetrazine, and the like.

The term “heterocycle”, unless specifically stated otherwise, refers to single and multi-cyclic non-aromatic ring systems in which at least one of the ring members is other than carbon. Heterocycle includes pyrrolidine, piperidine, piperazine, morpholine, azetidine, tetrahydropyran, tetrahydrofuran, dioxane, and the like.

The term “amine” unless specifically stated otherwise includes primary, secondary and tertiary amines.

The term “halogen” includes fluorine, chlorine, bromine and iodine atoms.

The term “oxide” of heteroaryl groups is used in the ordinary well-known chemical sense and includes, for example, N-oxides of nitrogen heteroatoms.

Compounds described herein contain one or more double bonds and may thus give rise to cis/trans isomers as well as other conformational isomers. The present invention includes all such possible isomers as well as mixtures of such isomers.

Compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above compounds of the invention may be shown without a definitive stereochemistry at certain positions. The present invention includes all stereoisomers of the compounds of the invention and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The term “optionally substituted” is intended to include both substituted and unsubstituted. Thus, for example, optionally substituted aryl can represent a pentafluorophenyl or a phenyl ring. Further, the substitution can be made at any of the groups. For example, substituted aryl(C₁₋₆)alkyl includes substitution on the aryl group as well as substitution on the alkyl group.

The term “polycyclic ring” means more than 3 fused rings and includes carbon as ring atoms. The polycyclic ring can be saturated or unsaturated. The polycyclic ring can be unsubstituted, singly substituted or, if possible, multiply substituted, with substituent groups in any possible position. The individual rings may or may not be of the same type. Examples of polycyclic rings include adamantane, bicyclooctane, norbornane and bicyclononanes.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

“Pharmaceutically acceptable non-toxic acids”, including inorganic and organic acids, salts prepared from, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

The pharmaceutical compositions of the present invention comprise a compound represented of the invention (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of the invention, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

The pharmaceutical compositions of the present invention comprise a compound of the invention (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles and the like. Further, the compositions can be in a form suitable for use in transdermal devices. These formulations may be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, may also be prepared in powder or liquid concentrate form.

A formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms can generally contain between from about 1 mg to about 1000 mg of the active ingredient.

The conditions recited herein can be treated or prevented by the administration of from about 0.01 mg to about 140 mg of the instant compounds per kilogram of body weight per day.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy. For example, inflammatory pain may be effectively treated by the administration of from about 0.01 mg to about 75 mg of the present compound per kilogram of body weight per day, or alternatively about 0.5 mg to about 3.5 g per patient per day. Neuropathic pain may be effectively treated by the administration of from about 0.01 mg to about 125 mg of the present compound per kilogram of body weight per day, or alternatively about 0.5 mg to about 5.5 g per patient per day.

It is understood that compounds of this invention can be administered at prophylactically effective dosage levels to prevent the above-recited conditions, as well as to prevent other conditions mediated through CB2 receptor.

The Compounds of the invention may be used with other therapeutic agents such as those described below. Such other therapeutic agent(s) may be administered prior to, simultaneously with, or following the administration of the cannabinoid receptor modulators in accordance with the invention.

Compounds of the invention may be used in combination with other drugs that are used in the treatment/prevention/suppression or amelioration of the diseases or conditions for which compounds of the invention are useful. Such other drugs may be administered, by a route and in an amount commonly used therefore, contemporaneously or sequentially with a compound of the invention. When a compound of the invention is used contemporaneously with one or more other drugs, a pharmaceutical composition containing such other drugs in addition to the compound of the invention is preferred. Accordingly, the pharmaceutical compositions of the present invention include those that also contain one or more other active ingredients, in addition to a compound of the invention. Examples of active ingredients that may be combined with a compound of the invention, either administered separately or in the same pharmaceutical compositions, include, but are not limited to: (1) non-steroidal anti-inflammatory agents, such as ibuprofen and naproxen; (2) COX-2 inhibitors, such as Celebrex and Arcoxia; (3) bradykinin B1 receptor antagonists; (4) sodium channel blockers and antagonists; (5) nitric oxide synthase (NOS) inhibitors; (6) glycine site antagonists; (7) potassium channel openers; (8) AMPA/kainate receptor antagonists; (9) calcium channel antagonists; (10) GABA-A receptor modulators (e.g., a GABA-A receptor agonist); (11) matrix metalloprotease (MMP) inhibitors; (12) thrombolytic agents; (13) opioids such as morphine; (14) neutrophil inhibitory factor (NIF); (15) L-Dopa; (16) carbidopa; (17) levodopa/carbidopa; (18) dopamine agonists such as bromocriptine, pergolide, pramipexole, ropinirole; (19) anticholinergics; (20) amantadine; (21) carbidopa; (22) catechol O-methyltransferase (“COMT”) inhibitors such as entacapone and tolcapone; (23) Monoamine oxidase B (“MAO-B”) inhibitors; (24) opiate agonists or antagonists; (25) 5HT receptor agonists or antagonists; (26) NMDA receptor agonists or antagonists; (27) NK1 antagonists; (28) selective serotonin reuptake inhibitors (“SSRI”) and/or selective serotonin and norepinephrine reuptake inhibitors (“SSNRI”); (29) tricyclic antidepressant drugs, (30) norepinephrine modulators; (31) lithium; (32) valproate; and (33) neurontin (gabapentin).

Additional examples of active ingredients that may be combined with a compound of the invention, either administered separately or in the same pharmaceutical compositions, include, but are not limited to: (34) cyclosporin (e.g., cyclosporin A); (35) CTLA4-Ig, antibodies such as anti-ICAM-3, anti-IL-2 receptor (Anti-Tac), anti-CD45RB, anti-CD2, anti-CD3 (OKT-3), anti-CD4, anti-CD80, anti-CD86, and monoclonal antibody OKT3; (36) agents blocking the interaction between CD40 and gp39, such as antibodies specific for CD40 and/or gp39 (i.e., CD154); (37) fusion proteins constructed from CD40 and gp39 (CD40Ig and CD8gp39), (38) inhibitors, such as nuclear translocation inhibitors of NF-kappa B function, such as deoxyspergualin (DSG); (38) steroids such as prednisone or dexamethasone; (39) gold compounds; (40) antiproliferative agents such as methotrexate, FK506 (tacrolimus, Prograf), mycophenolate mofetil; (41) cytotoxic drugs such as azathiprine and cyclophosphamide; (42) TNF-α. inhibitors such as tenidap; (43) anti-TNF antibodies or soluble TNF receptor such as etanercept (Enbrel); (44) rapamycin (sirolimus or Rapamune); (45) leflunomide (Arava); (46) anticytokines such as antilL-4 or IL-4 receptor fusion proteins and PDE 4 inhibitors such as Ariflo, and (47) the PTK inhibitors disclosed in the following U.S. patent applications, incorporated herein by reference in their entirety: Ser. No. 09/097,338, filed Jun. 15, 1998; Ser. No. 09/094,797, filed Jun. 15, 1998; Ser. No. 09/173,413, filed Oct. 15, 1998; and Ser. No. 09/262,525, filed Mar. 4, 1999. See also the following documents and references cited therein and incorporated herein by reference: Hollenbaugh, D., Et Al, “Cleavable CD40Ig Fusion Proteins and the Binding to Sgp39”, J. Immunol. Methods (Netherlands), 188(1), pp. 1-7 (Dec. 15, 1995); Hollenbaugh, D., et al, “The Human T Cell Antigen Gp39, A Member of the TNF Gene Family, Is a Ligand for the CD40 Receptor: Expression of a Soluble Form of Gp39 with B Cell Co-Stimulatory Activity”, EMBO J. (England), 11(12), pp. 4313-4321 (December 1992); and Moreland, L. W. et al., “Treatment of Rheumatoid Arthritis with a Recombinant Human Tumor Necrosis Factor Receptor (P75)-Fc Fusion Protein,” New England J. of Medicine, 337(3), pp. 141-147 (1997).

Thus, compounds of the invention may be useful as analgesics. For example they may be useful in the treatment of chronic inflammatory pain (e.g. pain associated with rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea.

Compounds of the invention may be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them.

Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and laminating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Compounds of the invention may also be useful in the treatment of inflammation, for example in allergies, asthma, autoimmune diseases such as transplant rejection (e.g., kidney, heart, lung, liver, pancreas, skin; host versus graft reaction (HVGR), graft versus host reaction (GVHR) etc.), rheumatoid arthritis, and amyotrophic lateral sclerosis, T-cell mediated autoimmune diseases such as multiple sclerosis, psoraiasis and Sjogren's syndrome, Type II inflammatory diseases such as vascular inflammation (including vasculitis, arteritis, atherosclerosis and coronary artery disease), diseases of the central nervous system such as stroke, pulmonary diseases such as bronchitis obliteraus and primary pulmonary hypertension, and solid, delayed Type IV hypersensitivity reactions, and hematologic malignancies such as leukemia and lymphomas.

Compounds of the invention may also be useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, motor neuron disease); vascular dementia (including multi-infarct dementia); as well as dementia associated with intracranial space occupying lesions; trauma; infections and related conditions (including HIV infection); dementia in Parkinson's disease; metabolism; toxins; anoxia and vitamin deficiency; and mild cognitive impairment associated with ageing, particularly Age Associated Memory Impairment. The compounds may also be useful for the treatment of amyotrophic lateral sclerosis (ALS) and neuroinflammation.

Compounds of the invention may also be useful in the treatment of psychiatric disease for example schizophrenia, depression (which term is used herein to include bipolar depression, unipolar depression, single or recurrent major depressive episodes with or without psychotic features, catatonic features, melancholic features, atypical features or postpartum onset, seasonal affective disorder, dysthymic disorders with early or late onset and with or without atypical features, neurotic depression and social phobia, depression accompanying dementia for example of the Alzheimer's type, schizoaffective disorder or the depressed type, and depressive disorders resulting from general medical conditions.

Compounds of the invention may also be useful in the treatment of cancer, including but not limited to adenomas, meningiomas, glioblastomas and melanoma.

The preferred uses of CB2 agonists are for the treatment of pain and inflammatory conditions. Pain is selected from inflammatory pain, visceral pain, cancer pain, neuropathic pain, lower back pain, muscular skeletal, post operative pain, acute pain, migraine and inflammatory pain associated with rheumatoid arthritis or osteoarthritis. Indications associated with inflammation include allergies, asthma, multiple sclerosis, vasculitis, arthritis, atherosclerosis and coronary artery disease.

Compounds of the invention are effective for treating and preventing pain, osteoarthritis, atherosclerosis, Multiple Sclerosis, Alzheimer's, and respiratory diseases.

Respiratory diseases for which the compounds of the invention are useful include but are not limited to chronic pulmonary obstructive disorder, emphysema, asthma, and bronchitis. Compounds of the invention are also useful in the treatment and prevention of indications disclosed in European Patent Documents Nos. EP 0570920 and EP 0444451; International Publications Nos. WO 97/29079, WO 99/02499, WO 98/41519, and WO 9412466; U.S. Pat. Nos. 4,371,720, 5,081,122, 5,292,736, and 5,013,387; and French Patent No. FR 2735774.

The compounds of the invention stimulate inhibitory pathways in cells, particularly in leukocytes, lung epithelial cells, or both, and are thus useful in treating respiratory diseases. “Leukocyte activation” is defined herein as any or all of cell proliferation, cytokine production, adhesion protein expression, and production of inflammatory mediators. “Epithelial cell activation” is defined herein as the production of any or all of mucins, cytokines, chemokines, and adhesion protein expression.

The Compounds of the invention are expected to block the activation of lung epithelial cells by moieties such as allergic agents, inflammatory cytokines or smoke, thereby limiting release of mucin, cytokines, and chemokines. Another preferred embodiment of the present invention comprises use of novel cannabinoid receptor modulator compounds to treat respiratory disease wherein the compounds selectively inhibit lung epithelial cell activation.

Thus, Compounds of the invention, in treating leukocyte activation-associated disorders are useful in treating a range of disorders such as: transplant (such as organ transplant, acute transplant, xenotransplant or heterograft or homograft (such as is employed in burn treatment)) rejection; protection from ischemic or reperfusion injury such as ischemic or reperfusion injury incurred during organ transplantation, myocardial infarction, stroke or other causes; transplantation tolerance induction; arthritis (such as rheumatoid arthritis, psoriatic arthritis or osteoarthritis); multiple sclerosis; respiratory and pulmonary diseases including but not limited to chronic obstructive pulmonary disease (COPD), emphysema, bronchitis, and acute respiratory distress syndrome (ARDS); inflammatory bowel disease, including ulcerative colitis and Crohn's disease; lupus (systemic lupus erythematosis); graft vs. host disease; T-cell mediated hypersensitivity diseases, including contact hypersensitivity, delayed-type hypersensitivity, and gluten-sensitive enteropathy (Celiac disease); psoriasis; contact dermatitis (including that due to poison ivy); Hashimoto's thyroiditis; Sjogren's syndrome; Autoimmune Hyperthyroidism, such as Graves' Disease; Addison's disease (autoimmune disease of the adrenal glands); Autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; glomerulonephritis; serum sickness; uticaria; allergic diseases such as respiratory allergies (asthma, hayfever, allergic rhinitis) or skin allergies; scleracierma; mycosis fungoides; acute inflammatory and respiratory responses (such as acute respiratory distress syndrome and ishchemia/reperfusion injury); dermatomyositis; alopecia greata; chronic actinic dermatitis; eczema; Behcet's disease; Pustulosis palmoplanteris; Pyoderma gangrenum; Sezary's syndrome; atopic dermatitis; systemic schlerosis; and morphea. The term “leukocyte activation-associated” or “leukocyte-activation mediated” disease as used herein includes each of the above referenced diseases or disorders. In a particular embodiment, the compounds of the present invention are useful for treating the aforementioned exemplary disorders irrespective of their etiology. The combined activity of the present compounds towards monocytes, macrophages, T-cells, etc. may be useful in treating any of the above-mentioned disorders.

Exemplary non-respiratory cannabinoid receptor-mediated diseases include transplant rejection, rheumatoid arthritis, multiple sclerosis, inflammatory bowel disease, lupus, graft v. host disease, T-cell mediated hypersensitivity disease, psoriasis, Hashimoto's thyroiditis, Guillain-Barre syndrome, cancer, contact dermatitis, allergic rhinitis, and ischemic or reperfusion injury.

Compounds of the invention also inhibit the Fc gamma dependent production of TNF-α in human monocytes/macrophages. The ability to inhibit Fc gamma receptor dependent monocyte and macrophage responses results in additional anti-inflammatory activity for the present compounds. This activity is especially of value, for example, in treating inflammatory diseases such as arthritis or inflammatory bowel disease. In particular, the present compouilds are useful for treating autoimmune glomerulonephritis and other instances of glomerulonephritis induced by deposition of immune complexes in the kidney that trigger Fc gamma receptor responses leading to kidney damage.

Cannabinoid receptors may be expressed on gut epithelial cells and hence regulate cytokine and mucin production and may be of clinical use in treating inflammatory diseases related to the gut. Cannabinoid receptors are also expressed on lymphocytes, a subset of leukocytes. Thus, cannabinoid receptor modulators will inhibit B and T-cell activation, proliferation and differentiation. Thus, such compounds will be useful in treating autoimmune diseases that involve either antibody or cell mediated responses such as multiple sclerosis and lupus.

In addition, cannabinoid receptors regulate the Fc epsilon receptor and chemokine induced degranulation of mast cells and basophils. These play important roles in asthma, allergic rhinitis, and other allergic disease. Fc epsilon receptors are stimulated by IgE-antigen complexes. Compounds of the present invention inhibit the Fc epsilon induced degranulation responses, including the basophil cell line, RBL. The ability to inhibit Fc epsilon receptor dependent mast cell and basophil responses results in additional anti-inflammatory and anti-allergic activity for the present compounds. In particular, the present compounds are useful for treating asthma, allergic rhinitis, and other instances of allergic disease.

The utility of the compounds of the invention can be demonstrated by the following assay.

### Cyclic AMP Assay

Chinese Hamster Ovary cells (CHO) expressing human CB1 or human CB2 (3.3×10⁵ cells/ml) were preincubated for 15 min at room temperature with tested agonist and 3-isobutyl-1-methylxanthine (IBMX; 200 μM) in phosphate buffered saline containing 1 mg/ml BSA (assay buffer) followed by 30 min incubation with forskolin in a total volume of 10 μl. The optimal forskolin concentration for each cell line was established in a separate experiment and adjusted to stimulate 70% of maximal cAMP response. cAMP content was measured using an HTRF assay (CisBio) according to the manufacturer's two step protocol.

In this assay, compounds of the invention have IC₅₀s ranging from 1 nM to >17000 nM. The Examples below have IC₅₀s ranging from 1 nM to >1700 nM.

| | | | CB2 |
|---|---|---|---|
| | | | IC₅₀ |
| EXAMPLE | COMPOUND | NAME | (NM) |
| I-1 | 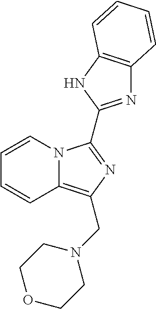 | 2-[1-(morpholin-4-ylmethyl) imidazo[1,5-a]pyridin-3-yl]- 1H-benzimidazole | 1263 |
| II-1 | 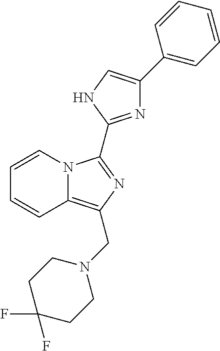 | 1-[(4,4-DIFLUOROPIPERIDIN-1- YL)METHYL]-3-(4-PHENYL-1H-IMIDAZOL- 2-YL)IMIDAZO[1,5-A]PYRIDINE | 178 |
| II-2 | 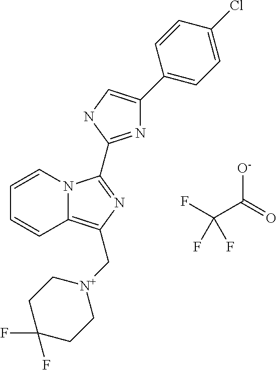 | 3-[4-(4-CHLOROPHENYL)-1H-IMIDAZOL-2- YL]-1-[(4,4-DIFLUOROPIPERIDIN-1- YL)METHYL]IMIDAZO[1,5-A]PYRIDINE | 199 |
| II-3 | 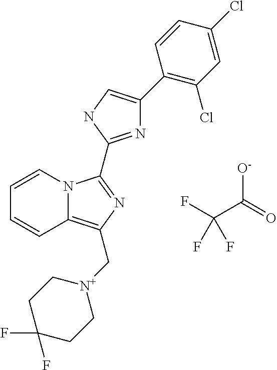 | 3-[4-(2,4-DICHLOROPHENYL)-1H- IMIDAZOL-2-YL]-1-[(4,4- DIFLUOROPIPERIDIN-1- YL)METHYL]IMIDAZO[1,5-A]PYRIDINE | 146 |
| II-4 | 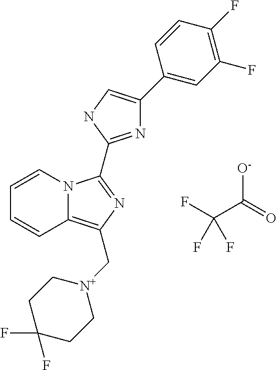 | 3-[4-(3,4-DIFLUOROPHENYL)-1H- IMIDAZOL-2-YL]-1-[(4,4- DIFLUOROPIPERIDIN-1-YL)METHYL] IMIDAZO[1,5-A]PYRIDINE | 171 |
| II-5 | 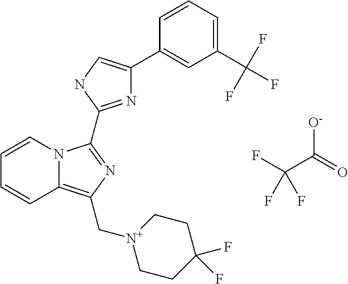 | 1-[(4,4-DIFLUOROPIPERIDIN-1- YL)METHYL]-3-{4-[3- (TRIFLUOROMETHYL)PHENYL]-1H- IMIDAZOL-2-YL}IMIDAZO [1,5-A]PYRIDINE | 165 |
| III-1 | 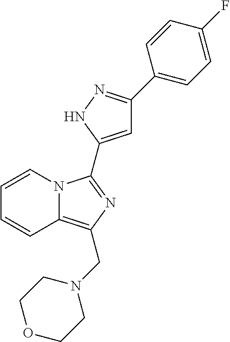 | 3-[3-(4-FLUOROPHENYL)-1H- PYRAZOL-5-YL]-1-(MORPHOLIN-4- YLMETHYL)IMIDAZO[1,5-A]PYRIDINE | 617 |
| III-3 | 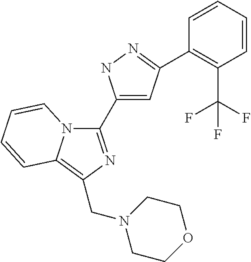 | 1-(MORPHOLIN-4-YLMETHYL)-3-{3-[3- (TRIFLUOROMETHYL)PHENYL]-1H- PYRAZOL-5-YL}IMIDAZO[1,5-A]PYRIDINE | 22 |
| IV-1 | 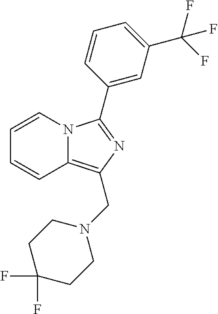 | 1-[(4,4-difluoropiperidin-l-yl)methyl]- 3-[3-(trifluoromethyl)phenyl] imidazo[1,5-a]pyridine | 17 |
| IV-4 | 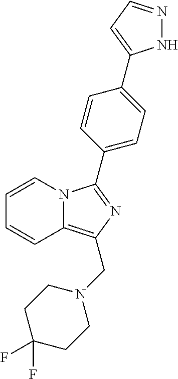 | 1-[(4,4-DIFLUOROPIPERIDIN-1- YL)METHYL]-3-[4-(1H-PYRAZOL-5- YL)PHENYL]IMIDAZO[1,5-A]PYRIDINE | 116 |
| IV-6 | 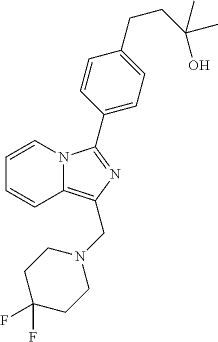 | 4-(4-{1-[(4,4-DIFLUOROPIPERIDIN-1- YL)METHYL]IMIDAZO[1,5-A]PYRIDIN-3- YL}PHENYL)-2-METHYLBUTAN-2-OL | 1351 |
| IV-7 | 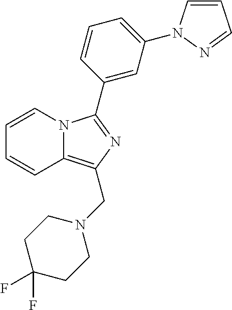 | 1-[(4,4-DIFLUOROPIPERIDIN-1- YL)METHYL]-3-[3-(1H-PYRAZOL-1- YL)PHENYL]IMIDAZO[1,5-A]PYRIDINE | 113 |

### Methods of Synthesis

Several methods for preparing the compounds of this invention are illustrated in the following Examples. Starting materials and the requisite intermediates are in some cases commercially available, or can be prepared according to literature procedures or as illustrated herein. All ¹H NMR spectra were obtained on instrumentation at field strength of 400 or 500 MHz.

The abbreviations used herein are as follows unless specified otherwise:

| | |
|---|---|
| ArB(OH)₂ | Aryl boronic acid |
| CDI | N,N′-Carbonyldiimidazole |
| DCE | 1,1-Dichloroethene |
| DIBAL or DIBAL-H | diisobutylaluminium hydride |
| DIPEA | N,N-diisopropyl-N-ethyl amine |
| DMF | dimethylformamide |
| EDC | 1,2-dichloroethane |
| HOAT | 1-Hydroxy-7-azabenzotriazole |
| HOBT | N-Hydroxybenzotriazole |
| LHMDS | lithium hexamethyldisilazane |
| NaBH(OAc)₃ | Sodium triacetoxyborohydride |
| TEA | Triethylamine |
| TFAA | Trifluoroacetic anhydride |

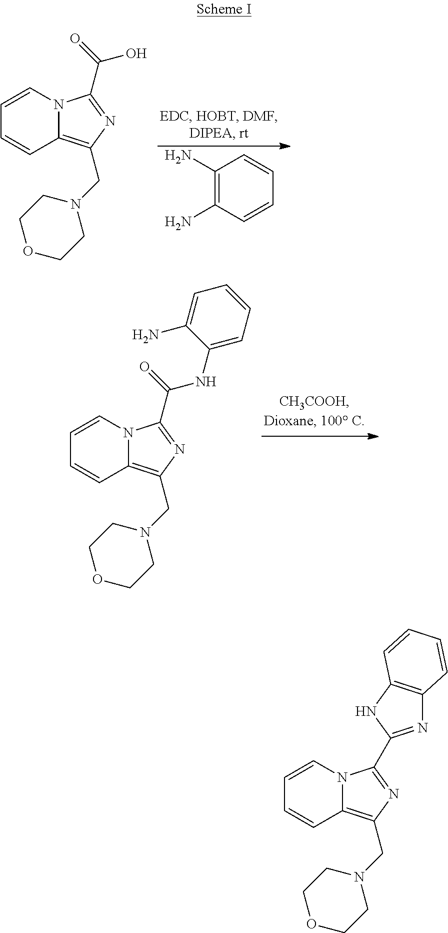

### Example I-1

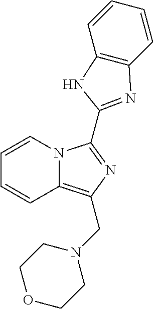

### 2-[1-(morpholin-4-ylmethyl)imidazo[1,5-a]pyridin-3-yl]-1H-benzimidazole

### Step A: N-(2-aminophenyl)-1-(morpholin-4-ylmethyl)imidazo[1,5-α]pyridine-3-carboxamide

A mixture of 1-(Morpholin-4-ylmethyl)imidazo[1,5-α]pyridine-3-carboxylic acid (0.062 g, 0.194 mmol), phenylenediamine (0.084 g, 0.233 mmol), EDC (0.074 g, 0.388 mmol, HOBT (0.059 g, 0.388 mmol), and DIPEA (0.134 mL, 0.776 mmol) in DMF (2 mL) was stirred @ rt o/n. The reaction was partitioned between saturated aqueous. NaHCO₃ and EtOAc. The aqueous phase was extracted 3× with EtOAc. The combined organic extracts were dried over Na₂SO₄, filtered, and concentrated. The resulting residue was purified by silica gel chromatography (0-4% MeOH in CH₂Cl₂). MS 265.1 (M-morpholine +1) ¹H NMR (500 MHz, CDCl₃) □ 9.48 (d, J=7.3 Hz, 1H), 9.01 (s, 1H), 7.74 (d, J=9.3, 1H), 7.37 (d, J=7.8 Hz 1H), 7.10 (td, J=7.7, 1.2 Hz 1H), 7.02-6.99 (m, 1H), 6.86-6.83 (m, 3H), 4.00 (bs, 2H), 3.85 (s, 2H), 3.74 (t, J=4.64 Hz, 4H), 2.55 (s, 4H).

### Step B: 2-[1-(morpholin-4-ylmethyl)imidazo[1,5-a]pyridin-3-yl]-1H-benzimidazole

To a mixture of N-(2-aminophenyl)-1-(morpholin-4-ylmethyl)imidazo[1,5-a]pyridine-3-carboxamide (0.025 g, 0.071 mmol) in Dioxane (1 mL) was added CH₃COOH (0.1 mL) The mixture was heated to 100° C. o/n. The mixture was cooled to rt, quenched with 2M Na₂CO₃ and extracted 3× with EtOAc. The combined organic extracts were dried over Na₂SO₄, filtered, and concentrated. The resulting residue was purified by silica gel chromatography (0-3% MeOH in CH₂Cl₂). HRMS: m/z found=334.1668 (M+1). ¹H NMR (500 MHz, CDCl₃) [symbol] (s, 1H), 9.80 (dd, J=7.2, 1.1 Hz, 1H), 7.84 (dd, J=6.1, 3.1 Hz, 2H), 7.69 (d, J=10.3 Hz, 1H), 7.46 (dd, J=6.1, 2.9), 7.31-7.27 (m, 2H), 6.97-6.94 (m, 1H), 6.89-6.86 (m, 1H), 3.88 (s, 2H), 3.73 (t, J=4.64 Hz, 4H), 2.55 (s, 4H).

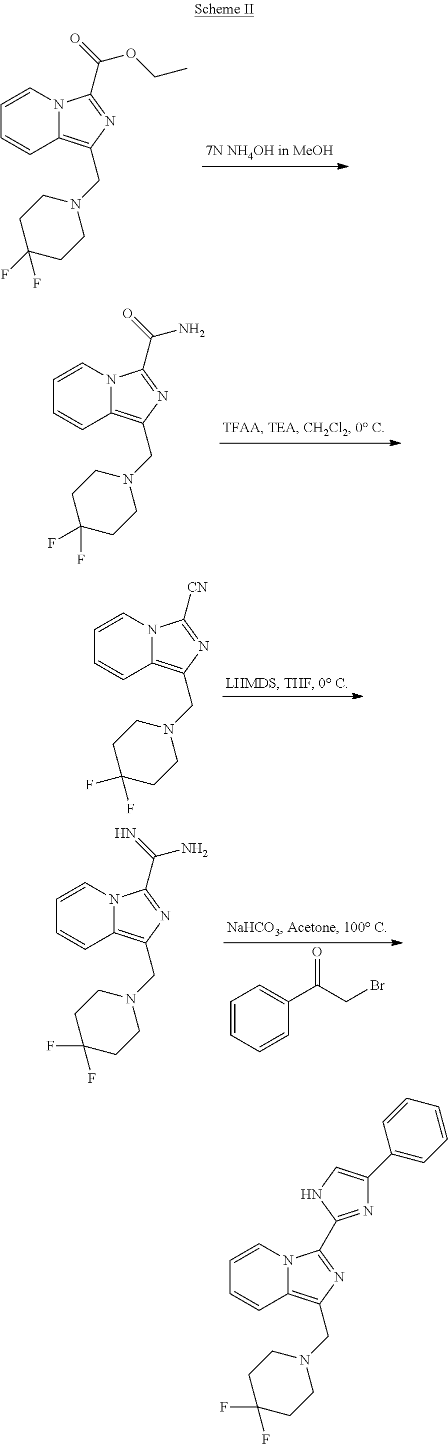

### Example II-1

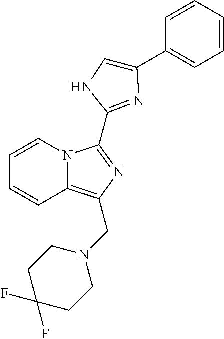

### 1-[(4,4-difluoropiperidin-1-yl)methyl]-3-(4-phenyl-1H-imidazol-2-yl)imidazo[1,5-a]pyridine

### Step A: 1-[(4,4-difluoropiperidin-1-yl)methyl]imidazo[1,5-a]pyridine-3-carboxamide

Dissolved 7.233 g 1 in 50 mL 7 N ammonia in methanol, then sealed r×n in a pressure tube and heated to 100 C in an oil bath for several hours. Cooled to room temperature, resaturated the solution with ammonia gas, then resealed and heated to 100 C overnight. Cooled to room temperature before opening. LC-MS indicates complete conversion of the ester starting material to the amide product, which crystallized on standing for several hours. The methanolic mixture was diluted with water, vacuum filtered, then washed with water. The resultant light yellow solid (title compound, was dried in vacuo overnight to yield the desired product as a (100% pure by HPLC). MS 174.0 (M-difluoropiperidine, carbocation signal). ¹H NMR (500 MHz, CDCl₃) [symbol] 9.38 (d, J=7.32 Hz, 1H), 7.83 (d, J=9.51 Hz, 1H), 7.09 (m, 1H), 6.94 (t, J=6.84 Hz, 1H), 3.90 (s, 2H), 2.67 (broad s, 4H), 1.98 (m, 4H).

### Step B: 1-[(4,4-difluoropiperidin-1-yl)methyl]imidazo[1,5-α]pyridine-3-carbonitrile

To a mixture of 1-[(4,4-difluoropiperidin-1-yl)methyl]imidazo[1,5-a]pyridine-3-carboxamide (0.540 g, 1.84 mmol) in CH₂Cl₂ (10 mL) was added TEA (0.537 mL, 3.85 mmol). The reaction was then cooled to 0° C. and TFAA (0.389 mL, 2.75 mmol) was added. The reaction was stirred for 1.5 hr @ 0° C., and then quenched with saturated aqueous NaHCO₃. The resulting mixture was extracted 3× with CH₂Cl₂. The combined organic extracts were dried over Na₂SO₄, filtered, and concentrated. The resulting residue was purified by silica gel chromatography (0-4% MeOH in CH2Cl2). MS 277.1 (M+1).

### Step C: 1-[(4,4-difluoropiperidin-1-yl)methyl]imidazo[1,5-α]pyridine-3-carboximidamide

To a mixture of 1-[(4,4-difluoropiperidin-1-yl)methyl]imidazo[1,5-α]pyridine-3-carbonitrile (0.257 g, 0.930 mmol) in THF (6 mL) @ 0° C. was added LHMDS (1.86 mL, 1M in THF). The mixture was stirred for 1 hr. It was quenched with 3 mL 1N HCl and stirred for 15 minutes. Then the reaction was neutralized with 3 mL NaOH. The resulting solution was diluted with 10 ml water and extracted 3× with EtOAc. The combined organic extracts were dried over Na₂SO₄, filtered, and concentrated. MS 173.1 (M-difluoropiperidine +1). ¹H NMR (500 MHz, CDCl₃) δ 9.68 (d, J=7.1 Hz, 1H), 7.64 (d, J=9.0 Hz, 1H), 6.93-6.90 (m, 1H), 6.78 (t, J=6.35 Hz 1H), 3.87 (s, 2H), 2.63 (s, 4H), 2.05-1.97 (m, 4H).

### Step D: 1-[(4,4-difluoropiperidin-1-yl)methyl]-3-(4-phenyl-1H-imidazol-2-yl)imidazo[1,5-a]pyridine

A mixture of 1-[(4,4-difluoropiperidin-1-yl)methyl]imidazo[1,5-α]pyridine-3-carboximidamide (0.050 g, 0.170 mmol), 2-bromoacetophenone (0.037 g, 0.188 mmol), and NaHCO3 (0.017 g, 0.205 mmol) in acetone (1.5 mL) was irradiated in the microwave @ 100° C. for 20 min. The reaction was partitioned between saturated aqueous NaHCO₃ and EtOAc. Extracted 3× with EtOAc and the combined organic extracts were dried over Na₂SO₄, filtered, and concentrated. Purified by acidic reverse phase HPLC (5% CH₃CN:95% H₂O+0.01% TFA to 55% CH₃—CN:45% H₂O+0.01% TFA). HRMS: m/z found=416.1654 (M+Na).

The following compounds were made according to Scheme I where intermediates in the scheme were modified according to literature methods.

### Examples II-2-II-5

| EXAMPLE | COMPOUND | NAME | MS (M + 1) |
|---|---|---|---|
| II-2 | 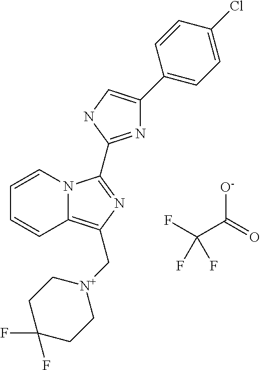 | 3-[4-(4-CHLOROPHENYL)- 1H-IMIDAZOL-2-YL]-1-[(4,4- DIFLUOROPIPERIDIN-1- YL)METHYL] IMIDAZO[1,5-A] PYRIDINE | 450.1265 (M + Na) |
| II-3 | 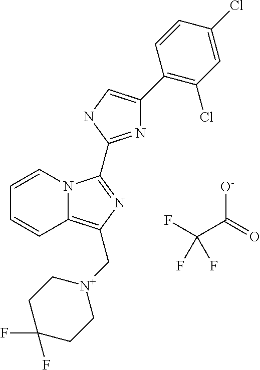 | 3-[4-(2,4- DICHLOROPHENYL)-1H- IMIDAZOL-2-YL]-1-[(4,4- DIFLUOROPIPERIDIN-1- YL)METHYL] IMIDAZO[1,5-A] PYRIDINE | 484.0880 (M + Na) |
| II-4 | 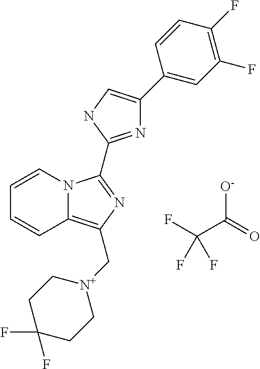 | 3-[4-(3,4- DIFLUOUROPHENYL)-1H- IMIDAZOL-2-YL]-1-[(4,4- DIFLUOROPIPERIDIN-1- YL)METHYL] IMIDAZO[1,5-A] PYRIDINE | 452.1469 (M + Na) |
| II-5 | 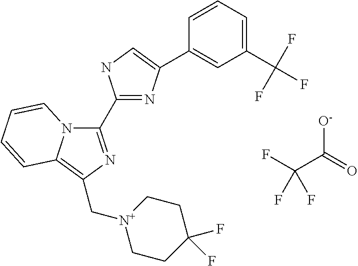 | 1-[(4,4- DIFLUOROPIPERIDIN- 1-YL)METHYL]-3-{4-[3- (TRIFLUOROMETHYL) PHENYL]-1H-IMIDAZOL- 2-YL}IMIDAZO[1,5-A] PYRIDINE | 484.1534 (M + Na) |

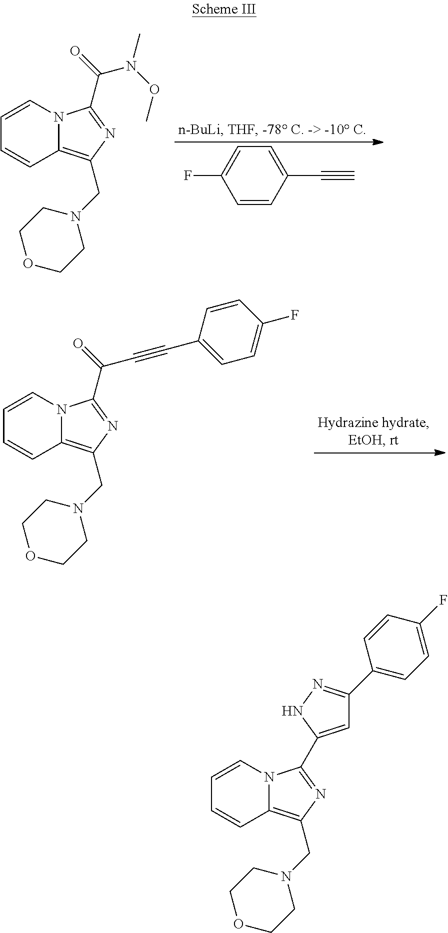

### Example III-1

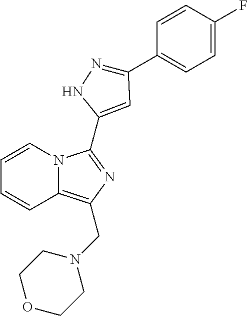

### 3-[3-(4-fluorophenyl)-1H-pyrazol-5-yl]-1-(morpholin-4-ylmethyl)imidazo[1,5-a]pyridine

### Step A: 3-(4-fluorophenyl)-1-[1-(morpholin-4-ylmethyl)imidazo[1,5-α]pyridin-3-yl]prop-2-yn-1-one

To a mixture of 1-ethynyl-4-fluorobenzene (0.020 g, 0.164 mmol) in THF (1 mL) under N2 @-78° C. was added n-BuLi (2.5M, 0.066 mL) dropwise. The mixture was stirred for 30 min @-78° C. and then N-Methoxy-N-methyl-1-(morpholin-4-ylmethyl)imidazo[1,5-a]pyridine-3-carboxamide (0.050 g, 0.164 mmol) in THF (1 mL) was added slowly. The mixture was stirred @-78° C. for 30 min and then warmed to −10° C. and stirred for 2 hr. The r×n was then quenched with saturated aqueous NH4Cl, then basified with NaHCO3 and extracted 3× with EtOAc. The combined organic extracts were dried over Na₂SO₄, filtered and concentrated. The resulting residue was purified by silica gel chromatography (0-1% MeOH in CH₂Cl₂). MS 277.1 (M-morpholine +1).

### Step B: 3-[3-(4-fluorophenyl)-1H-pyrazol-5-yl]-1-(morpholin-4-ylmethyl)imidazo[1,5-a]pyridine

To a mixture of 3-(4-fluorophenyl)-1-[1-(morpholin-4-ylmethyl)imidazo[1,5-α]pyridin-3-yl]prop-2-yn-1-one (0.024 g, 0.066 mmol) in EtOH (1 mL) was added hydrazine hydrate (80%) (0.013 mL, 0.264 mmol) at rt. The mixture was stirred @ rt for 2 hr. The mixture was concentrated and Purified by silica gel chromatography (0-4% MeOH in CH2Cl2). HRMS m/z 400.1539 (M+Na). ¹H NMR (500 MHz, CDCl₃) δ 10.71 (bs, 1H), 8.94 (s, 1H), 7.70-7.64 (m, 3H), 7.15 (t, J=8.7 Hz 3H), 6.82-6.79 (m, 1H), 6.73-6.70 (m, 1H), 3.89 (s, 2H), 3.72 (t, J=4.52 Hz, 4H), 2.56 (s, 4H).

The following compounds were made according to Scheme I where intermediates in the scheme were modified according to literature methods.

### Examples III-2-III-3

| EXAMPLE | COMPOUND | NAME | MS (M + 1) |
|---|---|---|---|
| III-2 | 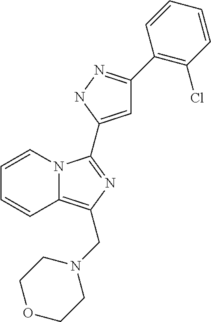 | 3-[3-(2-CHLOROPHENYL)- 1H-PYRAZOL-5-YL]- 1-(MORPHOLIN-4- YLMETHYL) IMIDAZO[1,5-A] PYRIDINE | 416.1245 (M + Na) |
| III-3 | 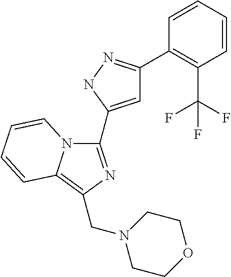 | 1-(MORPHOLIN-4- YLMETHYL)-3-{3-[3- (TRIFLUOROMETHYL) PHENYL]-1H-PYRAZOL- 5-YL}IMIDAZO[1,5-A] PYRIDINE | 450.1510 (M + Na) |

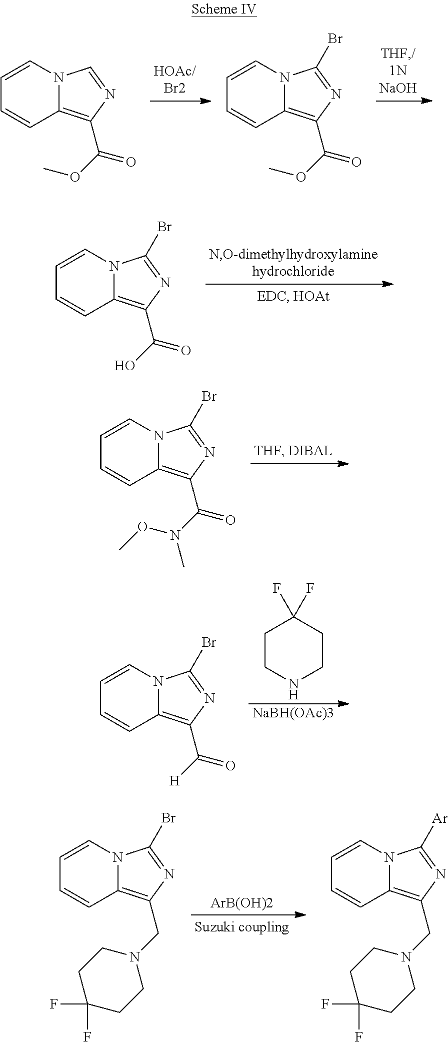

### Example IV-1

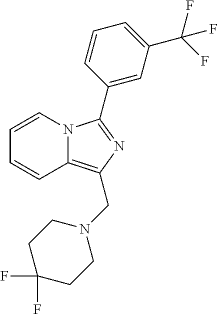

### 1-[(4,4-difluoropiperidin-1-yl)methyl]-3-[3-(trifluoromethyl)phenyl]imidazo[1,5-α]pyridine

### Step A: Methyl 3-bromoimidazo[1,5-α]pyridine-1-carboxylate

Dissolved 5.00 g methyl imidazo[1,5-α]pyridine-1-carboxylate in 24 mL HOAc, then added a solution of 1.5 mL bromine in 12 mL HOAc, dropwise, to the first solution. Stirred for an additional 5 min. after completion of the addition, then diluted with water and partially removed the solvent under reduced pressure at 50 C until a tan precipitate was evident. Removed from rotovap and cooled in an ice bath, then collected the tan solid by filtration, washing with water to yield the desired product (96% pure by HPLC). MS 255.0, 257.0 (M+H). ¹H NMR (500 MHz, CDCl₃) δ 8.20 (d, J=9.28 Hz, 1H), 8.05 (d, J=7.32 Hz, 1H), 7.20 (m, 1H), 6.93 (t, J=6.90 Hz, 1H), 3.99 (s, 3H).

### Step B: 3-bromoimidazo[1,5-α]oyridine-1-carboxylic acid

Dissolved 3.976 g methyl 3-bromoimidazo[1,5-α]pyridine-1-carboxylate in 50 mL THF. Added 24 mL 1 N NaOH and stirred for two days at 50 C in an oil bath. Rxn complete by LC-MS. Added 24 mL 1 N HCl to neutralize. A tan precipitate formed soon after the neutralization was complete. Vacuum filtered to collect the product, then washed with water. A second, darker brown precipitate formed in the filtrate. This was also collected and washed with water. Both solids were dried in vacuo to yield the desired product. MS 241.0, 243.0 (M+H). ¹H NMR (500 MHz, d₆-DMSO) δ 12.57 (bs, 1H), 8.31 (d, J=7.08 Hz, 1H), 8.06 (d, J=9.04 Hz, 1H), 7.33 (m, 1H), 7.09 (t, J=6.84 Hz, 1H) storfile wf013369

### Step C: 3-bromo-N-methoxy-N-methylimidazo[1,5-α]pyridine-1-carboxamide

Dissolved 2.94 g 3-bromoimidazo[1,5-α]pyridine-1-carboxylic acid, 1.35 g N,O-dimethylhydroxylamine hydrochloride, 2.26 g HOAT, and 3.06 g EDC.HCl in 40 mL DMF, then added 7.5 mL triethylamine and stirred overnight at room temperature. Reaction nearly complete by LC-MS. Added to 300 mL H₂O then extracted with EtOAc. Washed with 1:1 brine:water, then dried over Na₂SO₄. Filtered, concentrated, then purified on silica (20-40% EtOAc in hexanes) to yield the desired product (70% pure by HPLC; single compound by NMR). MS 284.0, 286.0 (M+H). ¹H NMR (500 MHz, CDCl₃) δ 8.28 (d, J=9.77 Hz, 1H), 7.99 (d, J=6.11 Hz, 1H), 7.09 (m, 1H), 6.87 (t, J=6.84 Hz, 1H), 3.90 (s, 3H), 3.59 (s, 3H).

### Step D: 3-bromoimidazo[1,5-α]pyridine-1-carbaldehyde

Dissolved 295 mg 3-bromo-N-methoxy-N-methylimidazo[1,5-α]pyridine-1-carboxamide in 10 mL THF, cooled to −78 C, then added 1.25 mL DIBAL-H (1 M in THF). At 1.5 hrs. mostly SM with some desbromo byproduct by LC-MS. Added 1.25 mL DIBAL-H. Still incomplete after an hour, but desired product mass is seen in major product. Added 0.5 mL DIBAL-H at 4.5 hrs. Rxn complete by 5 hrs. Added EtOAc to quench. Added Rochelle's salt as a saturated solution, then extracted with EtOAc. Dried over Na₂SO₄, filtered, concentrated, then purified on silica (20-40% EtOAc in hexanes) to yield the desired product as a light yellow solid (73% pure by HPLC; single compound by NMR). MS 225.0, 227.0 (M+H). ¹H NMR (500 MHz, CDCl₃) δ 10.05 (s, 1H), 8.30 (d, J=9.03 Hz, 1H), 8.10 (d, J=7.08 Hz, 1H), 7.33 (m, 1H), 7.04, (t, J=6.96 Hz, 1H).

### Step E: 3-bromo-1-[(4,4-difluoropiperidin-1-yl)methyl]imidazo[1,5-α]pyridine

Dissolved a mixture of 1.159 g 3-bromoimidazo[1,5-α]pyridine-1-carbaldehyde, 860 mg 4,4-difluoropiperidine hydrochloride, and 1.38 g sodium triacetoxyborohydride in 6 mL EDC (containing 2% HOAc) and stirred for several hours. Reaction complete by LC-MS. Added to sat. NaHCO₃, then extracted with CH₂Cl₂. Dried over Na₂SO₄, filtered, concentrated, then purified on silica (30-50% EtOAc in hexanes) to yield the desired product as a tan solid (100% pure by HPLC). MS 209.0, 211.0 (M-difluoropiperidine, carbocation signal). ¹H NMR (500 MHz, CDCl₃) δ 7.83 (d, J=7.33 Hz, 1H), 7.49 (d, J=9.27 Hz, 1H), 6.75 (m, 1H), 6.66 (t, J=6.84 Hz, 1H), 3.84 (s, 2H), 2.63 (broad s, 4H), 2.00 (m, 4H).

### Step F: 1-[(4,4-difluoropiperidin-1-yl)methyl]-3-[3-(trifluoromethyl)phenyl]imidazo[1,5-α]pyridine

Dissolved a mixture of 58.0 mg 3-bromo-1-[(4,4-difluoropiperidin-1-yl)methyl]imidazo[1,5-α]pyridine, 54 mg 4,4,5,5-tetramethyl-2-(3-trifluoromethylphenyl)-1,3,2-dioxaborolane, 17 mg PdCl₂dppf-dichloromethane adduct, and 158 mg cesium carbonate in 1.2 mL dioxane, then added 0.30 mL water. Heated to 150 C in a microwave reactor for 15 min. Rxn complete by LC-MS. Diluted with EtOAc, syringe filtered, then extracted from aq. NaHCO₃ with EtOAc. Dried over Na₂SO₄, concentrated, then separated on silica (30-50% EtOAc in hexanes) to yield the desired product as an orange oil. MS 275.2 (M-difluoropiperidine, carbocation signal). ¹H NMR (500 MHz, CDCl₃) δ 8.19 (d, J=7.08 Hz, 1H), 8.07 (s, 1H), 7.99 (d, J=7.33 Hz, 1H) 7.64 (m, 3H), 6.76 (m, 1H), 6.62 (t, J=6.84 Hz, 1H), 3.92 (s, 2H), 2.67 (bs, 1H), 2.02 (m, 4H).

The following compounds were made according to Scheme I where intermediates in the scheme were modified according to literature methods.

### Examples IV-2-IV-7

| EXAMPLE | COMPOUND | NAME | MS (M + 1) |
|---|---|---|---|
| IV-2 | 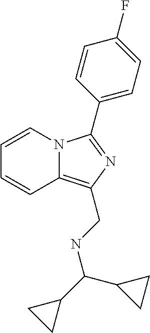 | 1,1-DICYCLOPROPYL-N-{[3- (4-FLUOROPHENYL)IMIDAZO [1,5-A]PYRIDIN-1-YL]METHYL} METHANAMINE | 358.1689 (M + Na) |
| IV-3 | 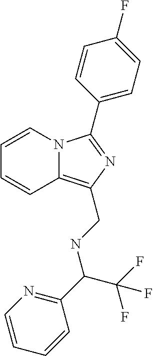 | 2,2,2-TRIFLUORO-N-{[3-(4- FLUOROPHENYL)IMIDAZO [1,5-A]PYRIDIN-1-YL] METHYL}-1-PYRIDIN-2- YLETHANAMINE | 423.1197 (M + Na) |
| IV-4 | 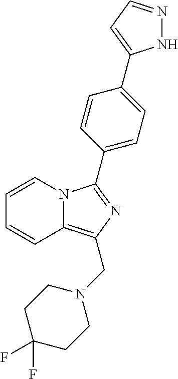 | 1-[(4,4-DIFLUOROPIPERIDIN- 1-YL)METHYL]-3-[4-(1H- PYRAZOL-5-YL)PHENYL] IMIDAZO[1,5-A]PYRIDINE | 273.2 (fragment) |
| IV-5 | 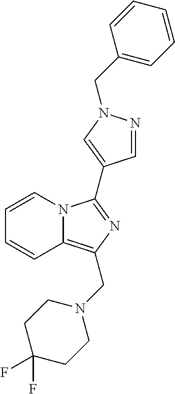 | 3-(1-BENZYL-1H-PYRAZOL- 4-YL)-1-[(4,4- DIFLUOROPIPERIDIN-1- YL)METHYL] IMIDAZO[1,5-A] PYRIDINE | 287.2 (fragment) |
| IV-6 | 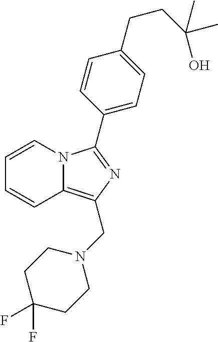 | 4-(4-{1-[(4,4- DIFLUOROPIPERIDIN-1- YL)METHYL]IMIDAZO[1,5- A]PYRIDIN-3-YL}PHENYL)- 2-METHYLBUTAN-2-OL | 414.2363 |
| IV-7 | 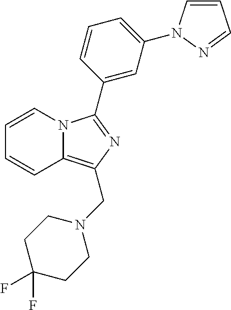 | 1-[(4,4- DIFLUOROPIPERIDIN-1- YL)METHYL]-3-[3- (1H-PYRAZOL-1- YL)PHENYL] IMIDAZO[1,5-A] PYRIDINE | 273.3 (fragment) |

What is claimed is:

1. A compound of Formula (I) and Formula (II): 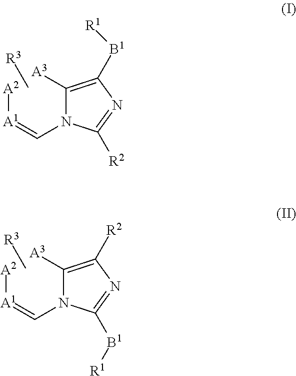
or a pharmaceutically acceptable salt thereof, wherein
A¹, A², and A³ are selected from the group consisting of:
(1) CH and
(2) N;
B¹ is aryl or heteroaryl;
R¹ and R² are independently selected from the group consisting of:
(1) H,
(2) halo,
(3) —CN,
(4) —CF₃,
(5) —C₁₋₆alkyl,
(6) —C(O)—NH—C₁₋₃alkyl-CF₃,
(7) —C(O)—NH—C₁₋₃alkyl-heteroaryl, wherein the heteroaryl is optionally mono, di or tri-substituted with substituents independently selected from R⁶,
(8) —C(O)-heteroaryl, wherein the heteroaryl is optionally mono, di or tri-substituted with substituents independently selected from R⁶,
(9) —C(O)-heterocycle, wherein the heterocycle is optionally mono, di- or tri-substituted with substituents independently selected from R⁶,
(10) —NR⁴R⁵,
(11) —C₁₋₄allyl-NR⁴R⁵,
(12) —C₁₋₄allyl-heterocycle, wherein the alkyl is optionally substituted with hydroxyl and wherein the heterocycle is optionally mono, di- or tri-substituted with substituents independently selected from R⁶,
(13) —C₁₋₄allyl-heteroaryl, optionally mono, di- or tri-substituted with substituents independently selected from R⁶,
(14) heterocycle, optionally mono, di- or tri-substituted with substituents independently selected from R⁶,
(15) heteroaryl, optionally mono, di- or tri-substituted with substituents independently selected from R⁶,
(16) aryl, optionally mono, di- or tri-substituted with substituents independently selected from R⁷,
(17) O-aryl, optionally mono, di- or tri-substituted with substituents independently selected from R⁷,
(18) —O-heteroaryl, optionally mono, di- or tri-substituted with substituents independently selected from R⁶,
(19) —NH-heteroaryl, wherein the heteroaryl is optionally mono, di- or tri-substituted with substituents independently selected R⁶, and
(20) —C₃₋₆cycloalkyl, optionally mono, di- or tri-substituted with substituents selected —CH₃, —O—C₁₋₆alkyl, hydroxy, —CH₂—OH, halo, —S(O)₂—CH₃, —C(O)—O—C₁₋₆alkyl, —C(O)—NHC₁₋₆allyl, —C(O)—N(C₁₋₆alkyl)₂, oxo, C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN;
R³ is selected from the group consisting of:
(1) H,
(2) halo,
(3) —C₁₋₄alkyl, optionally substituted with hydroxyl,
(4) —CF₃, and
(5) —OC₁₋₆allyl;
(6) —CN,
(7) —CHF₂,
(8) —O—CF₃,
(9) hydroxy,
(10) —S(O)₂—CH₃,
(11) —C(O)—O—C₁₋₆alkyl,
(12) —C(O)—NHC₁₋₆alkyl,
(13) —C(O)—N(C₁₋₆alkyl)₂,
(14) —C(O)—O—C(CH₃)₃,
(15) —C(O)-heteroaryl,
(16) —C₃₋₆cycloalkyl,
(17) —NH₂,
(18) —NH₂—C(O)—CF₃,
(19) —NH₂—C(O)—N(CH₃)₂,
(20) —NC(O)—NH₂,
(21) —NH—S(O)₂—CH₃,
(22) heteroaryl, optionally mono, di- or tri-substituted with substituents independently selected from R⁶, and
(23) aryl, optionally mono, di- or tri-substituted with substituents independently selected from R⁷;
R⁴ is selected from the group consisting of hydrogen and methyl;
R⁵ is selected from the group consisting of
(1) C₁₋₄allyl, optionally mono or di-substituted, with substituents independently selected from the group consisting of C₃₋₆cycloalkyl, —CF₃, heteroaryl, —C₁₋₃alkyl-CF₃, CH₃, hydroxy, tetrahydrofuran, and
(2) —C₁₋₃alkyl-C₃₋₆cycloallyl, wherein the cycloalkyl is optionally mono or di-substituted with substituents independently selected from the group consisting of halo, CF₃, CH₃, C₁₋₃alkyl, —OC₁₋₆alkyl, or
R⁴ and R⁵ are joined together so that along with the nitrogen to which they are attached, there is formed a heterocycle, wherein said heterocycle is optionally mono, di or tri-substituted with substituents independently selected from the group consisting of —OC₁₋₆allyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)₂, oxo, —C(O)—O—C(CH₃)₃, —C(O)-heteroaryl, —C₃₋₆cycloalkyl, —NH₂, —NH—C(O)—CF₃, —C(O)—NHC₁₋₆allyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆alkyl;
R⁶ is selected from the group consisting of —CN, —CH₃, —CF₃, —CHF₂, —CO₁₋₆alkyl, —O—CF₃, hydroxy, —CH₂—OH, halo, —S(O)₂—CH₃, —C(O)—O—C₁₋₆allyl, —C(O)—NHC₁₋₆alkyl, —C(O)—N(C₁₋₆allyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl;
R⁷ is selected from the group consisting of —CH₃, hydroxy, OH, halo, —S(O)₂—CH₃, C(O)—O—C₁₋₆alkyl, —C(O)—NHC₁₋₆allyl, —C(O)—N(C₁₋₆alkyl)₂, —(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆allyl;
provided that when the compound is of Formula (II), and B¹ is optionally substituted aryl, then R² is other than hydrogen, halo, cyano, optionally substituted aryl, optionally substituted heteroaryl or NR⁴R⁵ wherein both of R⁴ and R⁵ are hydrogen, or unsubstituted alkyl.

2. A compound according to claim 1 wherein
R¹ is selected from the group consisting of:
(1) H
(2) halo,
(3) —CN,
(4) —CF₃,
(5) —C₁₋₆alkyl,
(6) —C(O)—NH—C₁₋₃alkyl-CF₃,
(7) —C(O)—NH—C₁₋₃alkyl-heteroaryl, wherein the heteroaryl is optionally mono, di or tri-substituted with substituents independently selected from R⁶,
(8) heterocycle, optionally mono, di- or tri-substituted with substituents independently selected from R⁶,
(9) heteroaryl, optionally mono, di- or tri-substituted with substituents independently selected from R⁶,
(10) aryl, optionally mono, di- or tri-substituted with substituents independently selected from R⁷,
(11) O-aryl, optionally mono, di- or tri-substituted with substituents independently selected from R⁷,
(12) —O-heteroaryl, optionally mono, di- or tri-substituted with substituents independently selected from R⁶, and
(13) —NH-heteroaryl, wherein the heteroaryl is optionally mono, di- or tri-substituted with substituents independently selected R⁶.

3. A compound according to claim 1 wherein
R² is selected from a group consisting of:
(1) halo,
(2) —CF₃,
(3) —C(O)—NH—C₁₋₃alkyl-CF₃,
(4) —C(O)—NH—C₁₋₃allyl-heteroaryl, wherein the heteroaryl is optionally mono, di or tri-substituted with substituents independently selected from R⁶,
(5) —C(O)-heteroaryl, wherein the heteroaryl is optionally mono, di or tri-substituted with substituents independently selected from R⁶,
(6) —C(O)-heterocycle, wherein the heterocycle is optionally mono, di- or tri-substituted with substituents independently selected from R⁶,
(7) —NR⁴R⁵,
(8) —C₁₋₄alkyl-NR⁴R⁵,
(9) —C₁₋₄alkyl-heterocycle, wherein the alkyl is optionally substituted with hydroxyl and wherein the heterocycle is optionally mono, di- or tri-substituted with substituents independently selected from R⁶,
(10) —C₁₋₄alkyl-heteroaryl, optionally mono, di- or tri-substituted with substituents independently selected from R⁶,
(11) heterocycle, optionally mono, di- or tri-substituted with substituents independently selected from R⁶,
(12) heteroaryl, optionally mono, di- or tri-substituted with substituents independently selected from R⁶,
(13) aryl, optionally mono, di- or tri-substituted with substituents independently selected from R⁷,
(14) O-aryl, optionally mono, di- or tri-substituted with substituents independently selected from R⁷,
(15) —O-heteroaryl, optionally mono, di- or tri-substituted with substituents independently selected from R⁶,
(16) —NH-heteroaryl, wherein the heteroaryl is optionally mono, di- or tri-substituted with substituents independently selected R⁶, and
(17) —C₃₋₆cycloalkyl, optionally mono, di- or tri-substituted with substituents selected —CH₃, —O—C₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆allyl)₂, oxo, C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃, —CF₃ and —CN.

4. A compound according to claim 3 wherein
R² is selected from the group consisting of:
(1) —CF₃,
(2) —NR⁴R⁵,
(3) —C₁₋₄allyl-NR⁴R⁵,
(4) —C₁₋₄alkyl-heterocycle, wherein the alkyl is optionally substituted with hydroxyl and wherein the heterocycle is optionally mono, di- or tri-substituted with substituents independently selected from R⁶, and
(5) heterocycle, optionally mono, di- or tri-substituted with substituents independently selected from R⁶.

5. A compound according to claim 1 wherein
R³ is selected from the group consisting of:
(1) H,
(2) halo,
(3) —C₁₋₄alkyl, optionally substituted with hydroxyl,
(4) —CF₃, and
(5) —OC₁₋₆alkyl;
(6) —CN,
(7) —CHF₂,
(8) —O—CF₃,
(9) —S(O)₂—CH₃,
(10) —C(O)—O—C₁₋₆alkyl,
(11) —C(O)—NHC₁₋₆alkyl,
(12) —C(O)—N(C₁₋₆alkyl)₂,
(13) —C(O)—O—C(CH₃)₃,
(14) —C(O)-heteroaryl,
(15) —C₃₋₆cycloalkyl,
(16) —NH₂—C(O)—CF₃,
(17) —NH₂—C(O)—N(CH₃)₂,
(18) —NC(O)—NH₂, and
(19) —NH—S(O)₂—CH₃.

6. A compound according to claim 5 wherein
R³ is sleeted from a group consisting of:
(1) H,
(2) halo,
(3) —C₁₋₄alkyl, optionally substituted with hydroxyl,
(4) —CF₃, and
(5) —CN,
(6) —S(O)₂—CH₃,
(7) —C(O)—NHC₁₋₆alkyl,
(8) —C(O)—N(C₁₋₆alkyl)₂,
(9) —NH₂—C(O)—CF₃,
(10) —NH₂—C(O)—N(CH₃)₂,
(11) —NC(O)—NH₂, and
(12) —NH—S(O)₂—CH₃.

7. A compound according to claim 1 wherein
R⁴ and R⁵ are joined together so that along with the nitrogen to which they are attached, there is formed a heterocycle, wherein said heterocycle is optionally mono, di or tri-substituted with substituents independently selected from the group consisting of —OC₁₋₆alkyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)₂, oxo, —C(O)—O—C(CH₃)₃, —C(O)-heteroaryl, —C₃₋₆cycloalkyl, —NH₂, —NH—C(O)—CF₃, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆alkyl.

8. A compound according to claim 1 wherein
A¹, A², and A³ are selected from the group consisting of:
(1) CH and
(2) N;
B¹ is aryl or heteroaryl;
R¹ is selected from the group consisting of:
(1) H,
(2) halo,
(3) —CN,
(4) —CF₃,
(5) —C₁₋₆allyl,
(6) —C(O)—NH—C₁₋₃alkyl-CF₃,
(7) —C(O)—NH—C₁₋₃alkyl-heteroaryl, wherein the heteroaryl is optionally mono, di or tri-substituted with substituents independently selected from R⁶,
(8) heterocycle, optionally mono, di- or tri-substituted with substituents independently selected from R⁶,
(9) heteroaryl, optionally mono, di- or tri-substituted with substituents independently selected from R⁶,
(10) aryl, optionally mono, di- or tri-substituted with substituents independently selected from R⁷,
(11) O-aryl, optionally mono, di- or tri-substituted with substituents independently selected from R⁷,
(12) —O-heteroaryl, optionally mono, di- or tri-substituted with substituents independently selected from R⁶, and
(13) —NH-heteroaryl, wherein the heteroaryl is optionally mono, di- or tri-substituted with substituents independently selected R⁶_{;}
R² is selected from a group consisting of
(1) halo,
(2) —CF₃,
(3) —C(O)—NH—C₁₋₃alkyl-CF₃,
(4) —C(O)—NH—C₁₋₃alkyl-heteroaryl, wherein the heteroaryl is optionally mono, di or tri-substituted with substituents independently selected from R⁶,
(5) —C(O)-heteroaryl, wherein the heteroaryl is optionally mono, di or tri-substituted with substituents independently selected from R⁶,
(6) —C(O)-heterocycle, wherein the heterocycle is optionally mono, di- or tri-substituted with substituents independently selected from R⁶,
(7) —NR⁴R⁵,
(8) —C₁₋₄alkyl-NR⁴R⁵,
(9) —C₁₋₄alkyl-heterocycle, wherein the alkyl is optionally substituted with hydroxyl and wherein the heterocycle is optionally mono, di- or tri-substituted with substituents independently selected from R⁶,
(10) —C₁₋₄alkyl-heteroaryl, optionally mono, di- or tri-substituted with substituents independently selected from R⁶,
(11) heterocycle, optionally mono, di- or tri-substituted with substituents independently selected from R⁶,
(12) heteroaryl, optionally mono, di- or tri-substituted with substituents independently selected from R⁶,
(13) aryl, optionally mono, di- or tri-substituted with substituents independently selected from R⁷,
(14) O-aryl, optionally mono, di- or tri-substituted with substituents independently selected from R⁷,
(15) —O-heteroaryl, optionally mono, di- or tri-substituted with substituents independently selected from R⁶,
(16) —NH-heteroaryl, wherein the heteroaryl is optionally mono, di- or tri-substituted with substituents independently selected R⁶, and
(17) —C₃₋₆cycloalkyl, optionally mono, di- or tri-substituted with substituents selected —CH₃, —O—C₁₋₆alkyl, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, oxo, C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN;
R³ is selected from the group consisting of:
(1) H,
(2) halo,
(3) —C₁₋₄alkyl, optionally substituted with hydroxyl,
(4) —CF₃, and
(5) —OC₁₋₆alkyl;
(6) —CN,
(7) —CHF₂,
(8) —O—CF₃,
(9) —S(O)₂—CH₃,
(10) —C(O)—O—C₁₋₆alkyl,
(11) —C(O)—NHC₁₋₆alkyl,
(12) —C(O)—N(C₁₋₆alkyl)₂,
(13) —C(O)—O—C(CH₃)₃,
(14) —C(O)-heteroaryl,
(15) —C₃₋₆cycloallyl,
(16) —NH₂—C(O)—CF₃,
(17) —NH₂—C(O)—N(CH₃)₂,
(18) —NC(O)—NH₂, and
(19) —NH—S(O)₂—CH₃;
R⁴ is selected from the group consisting of hydrogen and methyl;
R⁵ is selected from the group consisting of:
(1) C₁₋₄alkyl, optionally mono or di-substituted, with substituents independently selected from the group consisting of C₃₋₆cycloallyl, —CF₃, heteroaryl, —C₁₋₃alkyl-CF₃, CH₃, hydroxy, tetrahydrofuran, and
(2) —C₁₋₃alkyl-C₃₋₆cycloalkyl, wherein the cycloalkyl is optionally mono or di-substituted with substituents independently selected from the group consisting of halo, CF₃, CH₃, C₁₋₃alkyl, —OC₁₋₆alkyl, or
R⁴ and R⁵ are joined together so that along with the nitrogen to which they are attached, there is formed a heterocycle, wherein said heterocycle is optionally mono, di or tri-substituted with substituents independently selected from the group consisting of —OC₁₋₆allyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)₂, oxo, —C(O)—O—C(CH₃)₃, —C(O)-heteroaryl, —C₃₋₆cycloalkyl, —NH₂, —NH—C(O)—CF₃, —C(O)—NHC₁₋₆allyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆allyl;
R⁶ is selected from the group consisting of —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆allyl;
R⁷ is selected from the group consisting of —CH₃, —O—C₁₋₆alkyl, hydroxy, —CH₂—OH, halo, —S(O)₂—CH₃, C(O)—O—C₁₋₆alkyl, —C(O)—NHC₁₋₆allyl, —C(O)—N(C₁₋₆allyl)₂, —(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl;
provided that when the compound is of Formula (II), and B¹ is optionally substituted aryl, then R² is other than hydrogen, halo, cyano, optionally substituted aryl, optionally substituted heteroaryl or NR⁴R⁵ wherein both of R⁴ and R⁵ are hydrogen, or unsubstituted alkyl.

9. A compound according to claim 1 wherein
A¹, A², and A³ are selected from the group consisting of:
(1) CH and
(2) N;
B¹ is aryl or heteroaryl;
R¹ is selected from the group consisting of:
(1) H,
(2) halo,
(3) —CN,
(4) —CF₃,
(5) —C₁₋₆alkyl,
(6) —C(O)—NH—C₁₋₃alkyl-CF₃,
(7) —C(O)—NH—C₁₋₃allyl-heteroaryl, wherein the heteroaryl is optionally mono, di or tri-substituted with substituents independently selected from R⁶,
(8) heterocycle, optionally mono, di- or tri-substituted with substituents independently selected from R⁶,
(9) heteroaryl, optionally mono, di- or tri-substituted with substituents independently selected from R⁶,
(10) aryl, optionally mono, di- or tri-substituted with substituents independently selected from R⁷,
(11) O-aryl, optionally mono, di- or tri-substituted with substituents independently selected from R⁷,
(12) —O-heteroaryl, optionally mono, di- or tri-substituted with substituents independently selected from R⁶, and
(13) —NH-heteroaryl, wherein the heteroaryl is optionally mono, di- or tri-substituted with substituents independently selected R⁶;
R² is selected from the group consisting of:
(1) —CF₃,
(2) —NR⁴R⁵,
(3) —C₁₋₄alkyl-NR⁴R⁵,
(4) —C₁₋₄alkyl-heterocycle, wherein the alkyl is optionally substituted with hydroxyl and wherein the heterocycle is optionally mono, di- or tri-substituted with substituents independently selected from R⁶, and
(5) heterocycle, optionally mono, di- or tri-substituted with substituents independently selected from R⁶_{;}
R³ is sleeted from a group consisting of:
(1) H,
(2) halo,
(3) —C₁₋₄allyl, optionally substituted with hydroxyl,
(4) —CF₃, and
(5) —CN,
(6) —S(O)₂—CH₃,
(7) —C(O)—NHC₁₋₆alkyl,
(8) —C(O)—N(C₁₋₆alkyl)₂,
(9) —NH₂—C(O)—CF₃,
(10) —NH₂—C(O)—N(CH₃)₂,
(11) —NC(O)—NH₂, and
(12) —NH—S(O)₂—CH₃;
R⁴ and R⁵ are joined together so that along with the nitrogen to which they are attached, there is formed a heterocycle, wherein said heterocycle is optionally mono, di or tri-substituted with substituents independently selected from the group consisting of —OC₁₋₆allyl, —NH—C(O)—O—C(CH₃)₃, hydroxy, —CH₃, —CF₃, —CH₂—OH, halo, —S(O)₂—CH₃, C(O)—O—C₁₋₆allyl, —C(O)—N(CH₃)₂, oxo, —C(O)—O—C(CH₃)₃, —C(O)-heteroaryl, —C₃₋₆cycloalkyl, —NH₂, —NH—C(O)—CF₃, —C(O)—NHC₁₋₆allyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆allyl;
R⁶ is selected from the group consisting of —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl;
R⁷ is selected from the group consisting of —CH₃, —O—C₁₋₆allyl, hydroxy, —CH₂—OH, halo, —S(O)₂—CH₃, C(O)—O—C₁₋₆alkyl, —C(O)—NHC₁₋₆allyl, —C(O)—N(C₁₋₆allyl)₂, —(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl.

10. A compound of Formula (Ia) and Formula (IIa), or a pharmaceutically acceptable salt thereof, wherein 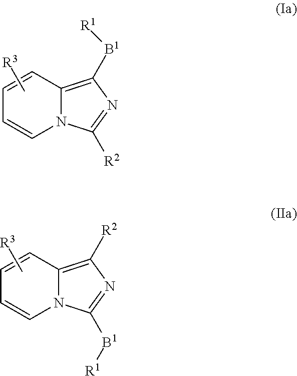
B¹ s aryl or heteroaryl;
R¹ and R² are independently selected from the group consisting of:
(1) H,
(2) halo,
(3) —CN,
(4) —CF₃,
(5) —C₁₋₆alkyl,
(6) —C(O)—NH—C₁₋₃alkyl-CF₃,
(7) —C(O)—NH—C₁₋₃allyl-heteroaryl, wherein the heteroaryl is optionally mono, di or tri-substituted with substituents independently selected from R⁶,
(8) —C(O)-heteroaryl, wherein the heteroaryl is optionally mono, di or tri-substituted with substituents independently selected from R⁶,
(9) —C(O)-heterocycle, wherein the heterocycle is optionally mono, di- or tri-substituted with substituents independently selected from R⁶,
(10) —NR⁴R⁵,
(11) —C₁₋₄alkyl-NR⁴R⁵,
(12) —C₁₋₄alkyl-heterocycle, wherein the alkyl is optionally substituted with hydroxyl and wherein the heterocycle is optionally mono, di- or tri-substituted with substituents independently selected from R⁶,
(13) —C₁₋₄alkyl-heteroaryl, optionally mono, di- or tri-substituted with substituents independently selected from R⁶,
(14) heterocycle, optionally mono, di- or tri-substituted with substituents independently selected from R⁶,
(15) heteroaryl, optionally mono, di- or tri-substituted with substituents independently selected from R⁶,
(16) aryl, optionally mono, di- or tri-substituted with substituents independently selected from R⁷,
(17) O-aryl, optionally mono, di- or tri-substituted with substituents independently selected from R⁷,
(18) —O-heteroaryl, optionally mono, di- or tri-substituted with substituents independently selected from R⁶,
(19) —NH-heteroaryl, wherein the heteroaryl is optionally mono, di- or tri-substituted with substituents independently selected R⁶, and
(20) —C₃₋₆cycloalkyl, optionally mono, di- or tri-substituted with substituents selected —CH₃, —O—C₁₋₆allyl, hydroxy, —CH₂—OH, halo, —S(O)₂—CH₃, —C(O)—O—C₁₋₆alkyl, —C(O)—NHC₁₋₆allyl, —C(O)—N(C₁₋₆alkyl)₂, oxo, C(O)—O—C(CH₃)₃, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN;
R³ is selected from the group consisting of:
(1) H,
(2) halo,
(3) —C₁₋₄alkyl, optionally substituted with hydroxyl,
(4) —CF₃, and
(5) —OC₁₋₆alkyl;
(6) —CN,
(7) —CHF₂,
(8) —O—CF₃,
(9) hydroxy,
(10) —S(O)₂—CH₃,
(11) —C(O)—O—C₁₋₆alkyl,
(12) —C(O)—NHC₁₋₆alkyl,
(13) —C(O)—N(C₁₋₆alkyl)₂,
(14) —C(O)—O—C(CH₃)₃,
(15) —C(O)-heteroaryl,
(16) —C₃₋₆cycloalkyl,
(17) —NH₂,
(18) —NH₂—C(O)—CF₃,
(19) —NH₂—C(O)—N(CH₃)₂,
(20) —NC(O)—NH₂,
(21) —NH—S(O)₂—CH₃,
(22) heteroaryl, optionally mono, di- or tri-substituted with substituents independently selected from R⁶, and
(23) aryl, optionally mono, di- or tri-substituted with substituents independently selected from R⁷;
R⁴ is selected from the group consisting of hydrogen and methyl;
R⁵ is selected from the group consisting of:
(1) C₁₋₄alkyl, optionally mono or di-substituted, with substituents independently selected from the group consisting of C₃₋₆cycloalkyl, —CF₃, heteroaryl, —C₁₋₃alkyl-CF₃, CH₃, hydroxy, tetrahydrofuran, and
(2) —C₁₋₃alkyl-C₃₋₆cycloalkyl, wherein the cycloalkyl is optionally mono or di-substituted with substituents independently selected from the group consisting of halo, CF₃, CH₃, C₁₋₃allyl, —OC₁₋₆alkyl, or
R⁴ and R⁵ are joined together so that along with the nitrogen to which they are attached, there is formed a heterocycle, wherein said heterocycle is optionally mono, di or tri-substituted with substituents independently selected from the group consisting of —OC₁₋₆allyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)₂, oxo, —C(O)—O—C(CH₃)₃, —C(O)-heteroaryl, —C₃₋₆cycloalkyl, —NH₂, —NH—C(O)—CF₃, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆alkyl;
R⁶ is selected from the group consisting of —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆alkyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl;
R⁷ is selected from the group consisting of —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl;
provided that when the compound is of Formula (II), and B¹ is optionally substituted aryl, then R² is other than hydrogen, halo, cyano, optionally substituted aryl, optionally substituted heteroaryl or NR⁴R⁵ wherein both of R⁴ and R⁵ are hydrogen, or unsubstituted alkyl.

11. A compound according to claim 10, or a pharmaceutically acceptable salt thereof, wherein
B¹ is aryl or heteroaryl;
R¹ is selected from the group consisting of:
(1) H,
(2) halo,
(3) —CN,
(4) —CF₃,
(5) —C₁₋₆alkyl,
(6) —C(O)—NH—C₁₋₃alkyl-CF₃,
(7) —C(O)—NH—C₁₋₃alkyl-heteroaryl, wherein the heteroaryl is optionally mono, di or tri-substituted with substituents independently selected from R⁶,
(8) heterocycle, optionally mono, di- or tri-substituted with substituents independently selected from R⁶,
(9) heteroaryl, optionally mono, di- or tri-substituted with substituents independently selected from R⁶,
(10) aryl, optionally mono, di- or tri-substituted with substituents independently selected from R⁷,
(11) O-aryl, optionally mono, di- or tri-substituted with substituents independently selected from R⁷,
(12) —O-heteroaryl, optionally mono, di- or tri-substituted with substituents independently selected from R⁶, and
(13) —NH-heteroaryl, wherein the heteroaryl is optionally mono, di- or tri-substituted with substituents independently selected R⁶;
R² is selected from the group consisting of:
(1) —CF₃,
(2) —NR⁴R⁵,
(3) —C₁₋₄allyl-NR⁴R⁵,
(4) —C₁₋₄alkyl-heterocycle, wherein the alkyl is optionally substituted with hydroxyl and wherein the heterocycle is optionally mono, di- or tri-substituted with substituents independently selected from R⁶, and
(5) heterocycle, optionally mono, di- or tri-substituted with substituents independently selected from R⁶_{;}
R³ is sleeted from a group consisting of:
(1) H,
(2) halo,
(3) —C₁₋₄allyl, optionally substituted with hydroxyl,
(4) —CF₃, and
(5) —CN,
(6) —S(O)₂—C₁₋₁₃,
(7) —C(O)—NHC₁₋₆allyl,
(8) —C(O)—N(C₁₋₆alkyl)₂,
(9) —NH₂—C(O)—CF₃,
(10) —NH₂—C(O)—N(CH₃)₂,
(11) —NC(O)—NH₂, and
(12) —NH—S(O)₂—CH₃;
R⁴ and R⁵ are joined together so that along with the nitrogen to which they are attached, there is formed a heterocycle, wherein said heterocycle is optionally mono, di or tri-substituted with substituents independently selected from the group consisting of —OC₁₋₆allyl, —NH—C(O)—O—C(CH₃)₃, hydroxy, —CH₃, —CF₃, —CH₂—OH, halo, —S(O)₂—CH₃, C(O)—O—C₁₋₆alkyl, —C(O)—N(CH₃)₂, oxo, —C(O)—O—C(CH₃)₃, —C(O)-heteroaryl, —C₃₋₆cycloallyl, —NH₂, —NH—C(O)—CF₃, —C(O)—NHC₁₋₆alkyl, —C(O)—N(C₁₋₆alkyl)₂, —NC(O)—NH₂, —NH—S(O)₂—CH₃, —O—CF₃, —S—CH₃, and wherein the heteroaryl portion of —C(O)-heteroaryl is optionally mono- di- or tri-substituted with substituents selected from the group consisting of halo, —CH₃, —CF₃, —CN and —O—C₁₋₆alkyl;
R⁶ is selected from the group consisting of —CN, —CH₃, —CF₃, —CHF₂, —OC₁₋₆alkyl, —O—CF₃, hydroxy, —CH₂—OH, halo, —S(O)₂—CH₃, —C(O)—O—C₁₋₆allyl, —C(O)—NHC₁₋₆alkyl, —C(O)—N(C₁₋₆alkyl)₂, —C(O)—O—C(CH₃)₃, —C(O)-heteroaryl, —C₃₋₆cycloalkyl, —NH₂, —NH₂—C(O)—CF₃, —NH₂—C(O)—N(CH₃)₂, —NC(O)—NH₂, and —NH—S(O)₂—CH₃, wherein the heteroaryl portion of —C(O)-heteroaryl, is optionally mono or di-substituted with substituents independently selected from halo, —CH₃, —CF₃, —CN and —OC₁₋₆alkyl;
R⁷ is selected from the group consisting of —CH₃, —O—C₁₋₆alkyl, hydroxy, —CH₂—OH, halo, —S(O)₂—CH₃, C(O)—O—C₁₋₆alkyl, —C(O)—NHC₁₋₆alkyl, —C(O)—N(C₁₋₆alkyl)₂, —(O)—O—C(CH₃)₃, C(O)-heteroaryl, —C₃₋₆cycloalkyl, —NH₂, —NH₂—C(O)—CF₃, —NH₂—C(O)—N(CH₃)₂, —NC(O)—NH₂, —NH—S(O)₂—CH₃, —O—CF₃, —CF₃ and —CN, wherein the heteroaryl portion of —C(O)-heteroaryl, is optionally mono or di-substituted with substituents independently selected from halo, —CH₃, —CF₃, —CN and —OC₁₋₆alkyl.

12. A compound according to claim 1 including:
2-[1-(morpholin-4-ylmethyl)imidazo[1,5-a]pyridin-3-yl]-1H-benzimidazole;
1-[(4,4-difluoropiperidin-1-yl)methyl]-3-(4-phenyl-1H-imidazol-2-yl) imidazo[1,5-a]pyridine;
3-[4-(4-chlorophenyl)-1H-imidazol-2-yl]-1-[(4,4-difluoropiperidin-1-yl)methyl]imidazo[1,5-a]pyridine;
3-[4-(2,4-dichlorophenyl)-1H-imidazol-2-yl]-1-[(4,4-difluoropiperidin-1-yl)methyl]imidazo[1,5-a]pyridine;
3-[4-(3,4-difluorophenyl)-1H-imidazol-2-yl]-1-[(4,4-difluoropiperidin-1-yl)methyl]imidazo[1,5-a]pyridine;
1-[(4,4-difluoropiperidin-1-yl)methyl]-3-{4-[3-(trifluoromethyl)phenyl]-1H-imidazol-2-yl}imidazo[1,5-a]pyridine;
3-[3-(4-fluorophenyl)-1H-pyrazol-5-yl]-1-(morpholin-4-ylmethyl) imidazo[1,5-a]pyridine;
3-[3-(2-chlorophenyl)-1H-pyrazol-5-yl]-1-(morpholin-4-ylmethyl) imidazo[1,5-a]pyridine;
1-(morpholin-4-ylmethyl)-3-{3-[3-(trifluoromethyl)phenyl]-1H-pyrazol-5-yl}imidazo[1,5-A]pyridine;
1-[(4,4-difluoropiperidin-1-yl)methyl]-3-[3-(trifluoromethyl)phenyl]imidazo[1,5-a]pyridine;
1,1-dicyclopropyl-N-{[3-(4-fluorophenyl) imidazo[1,5-a]pyridin-1-yl]methyl}methanamine;
2,2,2-trifluoro-N-{[3-(4-fluorophenyl) imidazo[1,5-a]pyridin-1-yl]methyl}-1-pyridin-2-ylethanamine;
1-[(4,4-difluoropiperidin-1-yl)methyl]-3-[4-(1H-pyrazol-5-yl)phenyl]imidazo[1,5-a]pyridine;
3-(1-benzyl-1H-pyrazol-4-yl)-1-[(4,4-difluoropiperidin-1-yl)methyl]imidazo[1,5-a]pyridine;
4-(4-{1-[(4,4-difluoropiperidin-1-yl)methyl]imidazo[1,5-a]pyridin-3-yl}phenyl)-2-methylbutan-2-ol;
1-[(4,4-difluoropiperidin-1-yl)methyl]-3-[3-(1H-pyrazol-1-yl)phenyl]imidazo[1,5-a]pyridine;
pharmaceutically acceptable salts and enantiomers thereof

13. A pharmaceutical composition comprising a compound of claim 1 and a pharmaceutically acceptable carrier.

14. A method of modulating the CB2 receptor in a patient in need of such modulation, comprising administering an effective amount of a compound according to claim 1.

15. A method of agonizing the CB2 receptor in a patient in need of such agonizing, comprising administering an effective amount of a compound according to claim 1.

16. A method of treating a disease mediated by agonizing the CB2 receptor in a patient in need of such treatment, comprising administering an effective amount of a compound according to claim 1.

17. A method of treating a disease selected from the group consisting of inflammatory pain, neuropathic pain, osteoporosis, atherosclerosis, immune disorders and arthritis comprising administering an effective amount of a compound according to claim 1.

18. A method according to claim 16, for the treatment of acute and chronic pain.

19. A method according to claim 17, for the treatment of inflammatory and neuropathic pain.
